SEQUENCE LISTING (1 sequence)

SEQ ID NO: 1 - protein, 687 aa, Human
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

(19) United States
(12) Patent Application Publication
(10) Pub. No.: US 2014/0079717 A1
(43) Pub. Date: Mar. 20, 2014

(54) Compositions and Methods to Prevent Cell Transformation and Cancer Metastasis

(71) Applicants: Rick Cerione, Ithaca, NY (US); Marc Antonyak, Ithaca, NY (US); Kristin Wilson Cerione, Ithaca, NY (US); Bo Li, Philadelphia, PA (US)

(72) Inventors: Rick Cerione, Ithaca, NY (US); Marc Antonyak, Ithaca, NY (US); Kristin Wilson Cerione, Ithaca, NY (US); Bo Li, Philadelphia, PA (US)

(73) Assignee: CORNELL UNIVERSITY, Ithaca, NY (US)

(21) Appl. No.: 13/985,740

(22) PCT Filed: Feb. 17, 2012
(86) PCT No.: PCT/US12/25603

§ 371 Date: Nov. 25, 2013

Related U.S. Application Data
(60) Provisional application No. 61/443,978, filed on Feb. 17, 2011.

(51) Int. Cl. G01N 33/68

(52) U.S. Cl. CPC G01N 33/6893
USPC 424/158.1; 435/7.4; 435/7.23

(57) ABSTRACT

Provided are methods for characterizing microvesicles or other membranous structures. The methods involve assaying samples for microvesicles or other membranous structures, and include in certain aspects determining the presence or absence of tissue transglutaminase (tTG) and/or cross-linked fibronection (FN). The microvesicles or other membranous structures can be separated from a sample using recombinant tTG or a derivative of it, or tTG or FN binding partners. Also provided are methods for inhibiting the transfer of cargo from microvesicles which contain tTG to one or more cells. This involves administering to the individual a tTG inhibitor, such as a cell-impermeable tTG inhibitor. Also provided are compositions which contain a population of microvesicles or other membranous structures, where the population is attached to tTG or a derivative thereof, or to tTG or an FN binding partner. Kits which contain reagents and other components for carrying out the method are also provided.

### CROSS-REFERENCE TO RELATED APPLICATIONS

This application claims priority to U.S. provisional patent application No. 61/443,978, filed Feb. 17, 2011, the disclosure of which is incorporated herein by reference.

### FIELD OF THE INVENTION

The present invention generally relates to diagnosis and therapy of cancer and more particularly to novel cancer biomarkers and therapies based on microvesicles that are shed from cancer cells.

### BACKGROUND OF THE INVENTION

Tumor progression involves the ability of cancer cells to communicate with each other and with neighboring normal cells in their microenvironment. Microvesicles (MVs) derived from human cancer cells have been receiving attention because of their apparent ability to participate in the horizontal transfer of signaling proteins between cancer cells and to contribute to their invasive activity. The release of MVs from different types of high-grade or aggressive forms of human cancer cells into their surroundings is becoming increasingly recognized as a feature of tumor biology, yet how these structures are generated and their importance in cancer progression are poorly understood. There is thus an ongoing and unmet need to develop compositions and methods that involve diagnostic methods and therapeutic interventions that exploit heretofore unrecognized aspects of the role of MVs in cancer. The present invention meets these needs.

### BRIEF SUMMARY OF THE INVENTION

The present invention is based in part on our discovery that there is a requirement for microvesicles (MVs) shed from cancer cells to contain tissue transglutaminase (tTG) to participate in MV-mediated transformation of normal cells. In this regard, we show that MVs shed by more than one type of human cancer cells can impart onto normal cells (fibroblasts and epithelial cells as representative non-cancer cells) a transformed phenotype, as evidenced by such qualities as enhanced survival capability and anchorage-independent growth. We also show that tTG is not adequate in and of itself to transform normal cells. Instead, it participates with a protein to which tTG binds and crosslinks (fibronectin (FN)) in the transformation of normal cells. Thus, identifying MVs that have the capacity to induce cellular transformation which is dependent upon the presence of tTG is one aspect of the invention. In another aspect, the invention provides methods for inhibiting induction of transformation of normal cells by modulating the effect of tTG that is associated with the microvesicles.

In one embodiment, the invention provides a method for characterizing microvesicles. The method comprises obtaining a sample which contains microvesicles and assaying the microvesicles for tTG. Based on assaying the MVs to determine whether or not they comprise tTG and/or cross-linked FN, the MVs are identified as tTG positive if tTG associated with the microvesicles is present in the sample, whereas the microvesicles are identified as tTG negative if tTG associated with the microvesicles is absent from the sample. In another embodiment the method comprises detecting the presence of tTG positive microvesicles. The method comprises obtaining a sample and assaying the sample to detect the presence of microvesicles that are associated with tTG. In other embodiments, the method comprises obtaining a sample which contains microvesicles and assaying the microvesicles to determine whether or not they comprise cross-linked FN. Based on assaying the MVs to determine whether or not they comprise cross-linked FN, the MVs are identified as cross-linked FN positive if cross-linked FN associated with the microvesicles is present in the sample, whereas the microvesicles are identified as cross-linked FN negative if cross-linked FN associated with the microvesicles is absent from the sample.

The method is suitable for analyzing any sample comprising MVs. In one embodiment, the sample comprises a liquid biological sample obtained or derived from an individual diagnosed with, suspected of having or is at risk for cancer. In one embodiment, the sample is obtained from an individual that is undergoing cancer therapy.

One aspect of the invention includes a method of diagnosing an individual as having circulating microvesicles that are tTG and/or cross-linked FN positive or tTG and/or cross-linked FN negative. This comprises assaying a sample obtained from the individual for tTG and/or cross-linked FN associated with the microvesicles, and identifying the individual as having circulating tTG and/or cross-linked FN associated microvesicles if tTG and/or cross-linked FN is present, respectively, and identifying the individual as not having circulating tTG and/or cross-linked FN associated microvesicles if tTG and/or cross-linked FN, respectively, is absent.

In various embodiments, assaying the microvesicles includes separating the microvesicles from the liquid biological sample by any suitable technique. In one embodiment, separating the MVs comprises capturing them using a binding partner. Any agent that can selectively bind to tTG or FN, or a complex comprising tTG and FN can be used. In certain embodiments, the binding partner is selected from FN, an anti-FN antibody or antibody binding fragment or derivative thereof, recombinant tTG and modified tTG, and fragments of tTG or modified tTG, an anti-tTG antibody or antibody binding fragment or derivative thereof, and combinations of the foregoing agents. Detection of the presence or absence of tTG can be performed using any suitable technique and reagents. In various embodiments, detection is performed using one or a combination of the aforementioned binding partners, wherein the binding partner is detectably labeled and/or is attached to a substrate.

In another aspect, the invention comprises a method for isolating membranous structures from a sample. This embodiment comprises providing a sample which may comprise the membranous structures and mixing the sample with tTG or a derivative thereof, and if the membranous structures are present in the sample, allowing formation of a complex of a membranous structure and the tTG or the derivative thereof. If present, the complex of the tTG and the membranous structure is separated from the rest of the sample.

Another aspect of the invention comprises inhibiting the transfer of cargo from microvesicles which comprise tTG to one or more cells in the individual. The method comprises administering to the individual a tTG inhibitor. The tTG inhibitor can be a cell-impermeable tTG inhibitor, such as a biologic agent, including but not necessarily limited to an antibody, or it can be a pharmaceutical agent, such as a small molecule tTG inhibitor.

Also provided by the invention is a composition comprising an isolated population of microvesicles, wherein the microvesicles comprise tTG, and wherein the isolated population of microvesicles is attached to a tTG or FN binding partner.

The invention also provides kits for detecting tTG positive microvesicles.

### BRIEF DESCRIPTION OF THE FIGURES

FIG. 1. MDAMB231 cells were analyzed by scanning electron microscopy SEM (left image) and immunofluorescent microscopy using rhodamine-conjugated phalloidin to detect F-actin (right image). Some of the largest MVs are indicated with arrows.

FIG. 2. Quantification of MV production by various cell lines cultured under serum-starved or EGF-stimulated conditions. Cells generating MVs were detected by labeling the samples with rhodamine-conjugated phalloidin. The data shown represents the mean±s.d. from three independent experiments.

FIG. 3. Images of cells from the experiment performed in FIG. 2. Some of the MVs are denoted with arrows.

FIG. 4. MDAMB231 cells transiently expressing a GFP-tagged form of the plasma membrane-targeting sequence from the Lyn tyrosine kinase (GFP-PM) were subjected to live-imaging fluorescent microscopy. Shown are a series of time-lapsed images taken in 2 minute intervals of a transfectant. The arrow denotes a MV that forms and is shed from a cell.

FIG. 5. Serum-deprived MDAMB231 cells that were either mock transfected or transfected with pEGFP (a plasmid encoding GFP) were lysed, and the MVs shed into the medium by the transfectants were isolated and lysed as well. The whole cell lysates (WCLs) and the MV lysates were then immunoblotted with antibodies against GFP, the MV-marker flotillin-2, and the cytosolic-specific-marker IκBα.

FIG. 6. Whole cell lysates (WCLs) of serum-starved MDAMB231 and U87 cells, as well as lysates of the MVs shed by these cells, were immunoblotted with antibodies against the MV-markers actin and flotillin-2, the cytosolic-specific-marker IκBα, and the activated (phospho)-EGF-receptor.

FIG. 7. Multiple sets of serum-deprived NIH3T3 fibroblasts were incubated with serum-free medium, medium containing 2% calf serum (CS), or medium supplemented with intact MVs derived from either MDAMB231 or U87 cells as indicated. One set of cells was lysed after being exposed to the various culturing conditions for the indicated lengths of time and then immunoblotted with antibodies that recognize the activated and total forms of AKT and ERK.

FIG. 8. Multiple sets of serum-deprived NIH3T3 fibroblasts were incubated with serum-free medium, medium containing 2% calf serum (CS), or medium supplemented with intact MVs derived from either MDAMB231 or U87 cells as indicated. Two additional sets of fibroblasts were evaluated for their abilities to undergo serum-deprivation-induced cell death. Data represents the mean±s.d. from at least three independent experiments.

FIG. 9. Multiple sets of serum-deprived NIH3T3 fibroblasts were incubated with serum-free medium, medium containing 2% calf serum (CS), or medium supplemented with intact MVs derived from either MDAMB231 or U87 cells as indicated. Two additional sets of fibroblasts were evaluated for their abilities to grow in low serum (2% CS). For the growth assays, the culturing medium (including the MVs) was replenished daily. Data represents the mean±s.d. from at least three independent experiments.

FIG. 10. NIH3T3 fibroblasts incubated without or with MVs derived from either MDAMB231 or U87 cells were subjected to anchorage-independent growth assays. The soft agar cultures were re-fed (including adding freshly prepared MVs) every third day. NIH3T3 cells expressing Cdc42 F28L was used as a positive control for these experiments. Data represents the mean±s.d. from at least three independent experiments.

FIG. 11. Images of the resulting colonies that formed in FIG. 10.

FIG. 12. Whole cell lysates (WCLs) of serum-starved MDAMB231 and U87 cells, as well as lysates of the MVs shed by these cells, were immunoblotted with several antibodies, including one against tTG.

FIG. 13. Top images—MDAMB231 cells immuno-stained with a tTG antibody. The boxed area was enlarged and arrows are used to denote certain MVs. Bottom images—a MDAMB231 cell co-stained with just the secondary antibody (left image) and Rhodamine-conjugated phalloidin to label the MVs (right image).

FIG. 14. Images of serum-starved U87 glioma cells and HeLa cervical carcinoma cells that were left untreated or stimulated with EGF for 15 minutes as indicated, and then immuno-stained with a tTG antibody. Pronounced MVs are denoted with arrows.

FIG. 15. Whole cell lysates (WCLs) of MDAMB231 cells ectopically expressing either GFP-only or GFP-tTG, as well as lysates of the MVs shed by these transfectants into their culturing medium, were immunoblotted with antibodies against GFP, the MV-marker flotillin-2, and the cytosolic-specific-marker IκBα.

FIG. 16. Fluorescent images of permeabilized and non-permeabilized samples of MDAMB231 cells stained with antibodies against tTG and the intracellular protein Rheb, and DAPI to label nuclei.

FIG. 17. Whole cell lysates (WCLs) of serum-starved MDAMB231 cells, as well as intact MVs generated by these cells treated without or with the tTG inhibitors T101 (cell-impermeable) or MDC (cell-permeable), were assayed for transamidation activity as readout by the incorporation of BPA into casein. The samples were then immunoblotted with antibodies against tTG, flotillin-2, and IκBα.

FIG. 18. The ability of MDAMB231 cell-derived MVs to induce cellular transformation requires the transfer of tTG from MVs to recipient cells. Extracts of serum-starved NIH3T3 fibroblasts that were incubated with serum-free medium or serum-free medium supplemented with MDAMB231 cell-derived MVs that had been pre-treated without or with the tTG inhibitor T101 for 30 minutes were immunoblotted with tTG and actin antibodies.

FIG. 19. The ability of MDAMB231 cell-derived MVs to induce cellular transformation requires the transfer of tTG from MVs to recipient cells. Extracts of serum-starved NIH3T3 fibroblasts that were incubated with serum-free medium or serum-free medium supplemented with MDAMB231 cell-derived MVs that had been pre-treated without or with the tTG inhibitor T101 for 30 minutes were assayed for transamidation activity as readout by the incorporation of BPA into lysate proteins. Data represents the mean±s.d. from at least three independent experiments.

FIG. 20. Cell death assays were performed on fibroblasts maintained in serum-free medium, 2% CS-medium, or serum-free medium containing MDAMB231 cell-derived MVs. Each culturing medium was further supplemented without or with the tTG inhibitors T101 (cell-impermeable) or MDC (cell-permeable) as indicated. Data represents the mean±s.d. from at least three independent experiments.

FIG. 21. Anchorage-independent growth assays were performed on fibroblasts incubated with MDAMB231 cell-derived MVs treated without or with T101, the RGD-peptide, or the control RGE-peptide. Data represents the mean±s.d. from at least three independent experiments.

FIG. 22. Lysates of NIH3T3 cells stably overexpressing vector alone or Myc-tTG were immunoblotted with Myc and actin antibodies, and assayed for transamidation activity as readout by the incorporation of BPA into lysate proteins.

FIG. 23. Cell death assays were performed on the NIH3T3 stable cell lines maintained in serum-free medium that was treated with T101, MDC, or 2% CS-medium, or was untreated. Data represents the mean±s.d. from at least three independent experiments.

FIG. 24. Anchorage-independent growth assays were performed on the NIH3T3 stable cell lines. Vector-control fibroblasts were incubated with MDAMB231 cell-derived MVs, as a positive control. Data represents the mean±s.d. from at least three independent experiments.

FIG. 25. Tumor formation assays were performed in which 5×10⁵ mitotically-arrested (using mitomycin-C) MDAMB231 cells (denoted as Mito-C-MDAMB231) expressing either control siRNA (siCont) or tTG siRNAs (denoted as siTG-1 or siTG-2) were subcutaneously injected singly, or combined with 5×10⁵ NIH3T3 fibroblasts, into nude mice. As controls, untreated MDAMB231 and NIH3T3 cells were injected into nude mice. The resulting tumors that formed for each condition were counted and the results shown in the table.

FIG. 26. Whole cell lysates (WCLs) of MDAMB231 and lysates of the MVs shed by these cells were either immunoblotted (Input), or subjected to immunoprecipitation using a tTG antibody (IP: tTG) and then immunoblotted, with FN, tTG, and actin antibodies. Note the detection of crosslinked FN in the MV lanes (FN dimer).

FIG. 27. Intact MVs collected from MDAMB231 or U87 cells were treated without or with T101 prior to being lysed. The MV extracts were then immunoblotted with FN and tTG antibodies. Note that the crosslinked forms of FN detected in the MV samples (FN dimer) were significantly reduced by T101 treatment.

FIG. 28. Lysates of fibroblasts that were incubated without or with MVs derived from MDAMB231 and U87 cells that had been pre-treated without or with T101 were immunoblotted with antibodies against FAK and ERK, or with antibodies that specifically recognize the activated forms of these protein kinases.

FIG. 29. Diagram depicting how MVs transform recipient cells. MVs containing tTG and fibronectin are generated and released from the surfaces of human cancer cells. The MVs can then be taken-up by, or directly alter the microenvironment of, neighboring normal cells, where the co-transfer of tTG and FN function cooperatively on the recipient cells to induce signaling events that promote cell survival and aberrant cell growth.

FIG. 30. MVs are constitutively released by MDAMB231 breast cancer cells into their culturing medium. MDAMB231 cells that were either mock transfected or transfected with a plasmid encoding GFP (pEGFP) were placed in serum-free medium for a day. The conditioned medium from the transfectants were collected and the intact MVs present in the medium were isolated and subjected to FACS analysis by gating for GFP-positive MVs that are between ˜1-3 μm in diameter. The results obtained when the MVs isolated from the mock transfected MDAMB231 cells were analyzed.

FIG. 31. MVs are constitutively released by MDAMB231 breast cancer cells into their culturing medium. MDAMB231 cells that were either mock transfected or transfected with a plasmid encoding GFP (pEGFP) were placed in serum-free medium for a day. The conditioned medium from the transfectants were collected and the intact MVs present in the medium were isolated and subjected to FACS analysis by gating for GFP-positive MVs that are between ˜1-3 μm in diameter. The results obtained when the MVs isolated from MDAMB231 cells transiently expressing GFP were analyzed.

FIG. 32. MDAMB231 cell-derived MVs transform recipient MCF10A mammary epithelial cells. Cell death assays were performed on MCF10A cells that were cultured for 3 days in serum-free medium, medium containing 2% fetal bovine serum (FBS), or serum-free medium supplemented with intact MVs derived from 5.0×10⁶ MDAMB231 cells. The data shown represent the mean±s.d. from three independent experiments.

FIG. 33. MDAMB231 cell-derived MVs transform recipient MCF10A mammary epithelial cells. Anchorage-independent growth assays were performed on MCF10A cells incubated with MVs derived from 5.0×10⁶ MDAMB231 cells treated without or with the tTG inhibitor T101. The culturing medium (including the MVs, T101, and AG1478) for the soft agar assays performed was replenished every third day for 12 days, at which time the colonies that formed were counted. The data shown represent the mean±s.d. from three independent experiments.

FIG. 34. MDAMB231 cell-derived MVs transform recipient MCF10A mammary epithelial cells. Anchorage-independent growth assays were performed on NIH3T3 fibroblasts incubated with U87 cell-derived MVs treated without or with the EGF receptor inhibitor AG1478. The culturing medium (including the MVs, T101, and AG1478) for the soft agar assays performed was replenished every third day for 12 days, at which time the colonies that formed were counted. The data shown represent the mean±s.d. from three independent experiments.

FIG. 35. Intact MDAMB231 cell-derived MVs were isolated, fixed, immuno-stained with a tTG antibody, and then processed for detection by SEM. Shown is a representative SEM image of a MV. Note the detection of tTG on the surface of the MV.

FIG. 36. The transamidation activity of a fixed concentration of purified recombinant tTG (1 μM) incubated with increasing concentrations of the tTG inhibitor T101 was assayed. The IC50 of T101 (dashed lines) was determined to be ˜1.5 μM. This experiment was repeated two additional times, with comparable results.

FIG. 37. Transamidation activity assays, as readout by the incorporation of BPA into lysate proteins, were performed on the cell extracts of MDAMB231 cells that had been cultured in medium supplemented without or with 200 μM T101 (a 133-fold greater concentration than the IC50 calculated for this inhibitor in B) for ˜10 hours prior to being washed extensively and then lysed (cell cultures). Equal amounts of a MDAMB231 cell extract were left untreated or were incubated with 10 μM T101 15 minutes before being subjected to a transamidation activity assay (cell extracts). Data are mean±s.d. from three independent experiments.

FIG. 38. Serum-starved MDAMB231 cells treated without or with T101, MDC, BFA, or Exo1, were immuno-stained with a tTG antibody. Shown are representative images of the cells exposed to the various inhibitors. Cells forming MVs are denoted with arrows.

FIG. 39. Serum-starved MDAMB231 cells treated without or with the tTG inhibitors T101 and MDC (left panel), transfected with either control siRNA (siCont) or two distinct tTG siRNAs (siTG-1 and siTG-2) (middle panel), or treated without or with the inhibitors of classical secretion, BFA and Exo1 (right panel), were lysed (WCLs) and the MVs released into the medium by the cells were also collected and lysed. The extracts were immunoblotted with antibodies against tTG, the MV-marker flotillin-2, and the cytosolic-specific-marker IκBα.

FIG. 40. Whole cell lysates (WCLs) of MDAMB231 cells ectopically expressing vector-only or Myc-tagged forms of wild-type tTG (TG WT), a transamidation-defective form of tTG (TG C277V), or a GTP-binding-defective form of tTG (TG R580L), as well as lysates of the MVs shed by the transfectants, were immunoblotted with antibodies against the Myc-tag, flotillin-2, and IκBα.

FIG. 41. Lysates of fibroblasts incubated for 30 minutes with U87 cell-derived MVs that had been pre-treated without or with the cell impermeable tTG inhibitor T101 were immunoblotted with tTG and actin antibodies.

FIG. 42. NIH3T3 cells incubated for 30 minutes with serum-free medium supplemented without or with intact MVs generated by either MDAMB231 or U87 cells were immuno-stained with a tTG antibody and rhodamine-conjugated phalloidin to detect actin. Shown are representative fluorescent images of the fibroblasts. Note that tTG is only detected in the fibroblasts that were incubated with cancer cell-derived MVs.

FIG. 43. Lysates of fibroblasts incubated for 30 minutes with U87 cell-derived MVs that had been pre-treated without or with T101 were assayed for transamidation activity as read-out by the incorporation of BPA into lysate proteins.

FIG. 44. Cell death assays were performed on fibroblasts maintained in serum-free medium, 2% CS-medium, or serum-free medium containing MVs derived from 5.0×10⁶ U87 glioma cells. Each culturing medium was either further supplemented with the cell-impermeable tTG inhibitor T101 or was untreated. The data shown represent the mean±s.d. from at least three independent experiments.

FIG. 45. The MVs shed from 5.0×10⁶ serum-starved MDAMB231 cells transfected with either control siRNA (siCont) or two different tTG siRNAs (siTG-1 and siTG-2) were collected and resuspended in serum-free DMEM. NIH3T3 cells plated in each well of a 6-well dish were then placed in serum-free medium or serum-free medium containing the different MV preparations for ˜35 hours, at which time the cell death rates of the different cell cultures were determined. The data shown represent the mean±s.d. from at least three independent experiments.

FIG. 46. The MVs shed from 5.0×10⁶ serum-starved MDAMB231 cells transfected with either control siRNA (siCont) or two different tTG siRNAs (siTG-1 and siTG-2) were collected and resuspended in serum-free DMEM. Anchorage-independent growth assays were performed on NIH3T3 fibroblasts incubated with the different MV preparations outlined above. The soft agar cultures were re-fed (including the addition of freshly prepared MVs) every third day for 12 days, at which time the colonies that formed were counted. The data shown represent the mean±s.d. from at least three independent experiments.

FIG. 47. Anchorage-independent growth assays were performed on control NIH3T3 fibroblasts or on fibroblasts incubated with MVs derived from 5.0×10⁶ U87 glioma cells treated with either T101, the RGD-peptide, the RGE-control-peptide, or untreated. The data shown represent the mean±s.d. from at least three independent experiments.

FIG. 48. Anchorage-independent growth assays were also performed on NIH3T3 cells stably expressing vector alone or an activated form of Cdc42 (Cdc42 F28L) treated with 200 μM T101 or untreated. Note that the ability of Cdc42 F28L to induce colony formation is insensitive to T101. The data shown represent the mean±s.d. from at least three independent experiments.

FIG. 49. T101 does not interfere with the ability of tTG to associate with FN in MDAMB231 cell- or U87 cell-derived MVs. Intact MVs collected from MDAMB231 or U87 cells were treated without or with T101 prior to being lysed. The MV extracts were then subjected to immunoprecipitation using a tTG antibody (IP: tTG). The resulting immunocomplexes were immunoblotted with FN and tTG antibodies.

FIG. 50. Microvesicles can be isolated from the conditioned medium of cancer cells using antibodies against tTG and fibronectin, or by using recombinant tTG. Serum-starved cultures of MDAMB231 breast cancer cells (231) and U87 brain tumor cells (U87) were lysed (WCL) and the medium from these cells collected (conditioned medium). Immunoprecipitations (IPs) were performed on medium using a tTG or fibronectin (FN) antibody bound to protein-G beads, as indicated. Purified, recombinant tTG bound to nickel beads (PPT: Rec. tTG) was also incubated with the conditioned medium. The complexes that precipitated with the antibodies or with the recombinant form of tTG, as well as samples of the whole cell lysates (WCL) from the cancer cells were subjected to Western blot analysis using the indicated antibodies. The presence of flotillin, a microvesicle marker, in the immunoprecipitation (IP) and the precipitation (PPT) lanes, indicates that antibodies against tTG and fibronectin, as well as the recombinant form of tTG, can capture microvesicles that have been shed into the medium by cancer cells.

FIG. 51. Recombinant tTG can associate with lipid vesicles lacking any protein content. Synthetic liposomes were prepared by extrusion and then equal amounts of this preparation were combined with either 5 μg recombinant tTG (tTG WT) or 5 μg bovine serum albumin (BSA). After a 15 minute incubation, the liposomes were pelleted by centrifugation, and the resulting supernatant (Sup) and liposome (Pellet) fractions were resolved by SDS-PAGE. The gel was then stained with Quick Blue to detect the proteins. A lane containing recombinant tTG (Rec. tTG WT) was included as a standard.

### DETAILED DESCRIPTION OF THE INVENTION

The present invention takes advantage of our discovery that MV-mediated transformation of normal cells is dependent in part on a requirement for the MVs to contain tTG. In particular, we demonstrate that MVs shed by different types of human cancer cells are capable of conferring onto normal fibroblasts and epithelial cells the transformed characteristics of cancer cells, such as anchorage-independent growth and enhanced survival capability, and that this requires the transfer of the protein crosslinking enzyme tTG to the cells. We further demonstrate that tTG is not sufficient to transform normal cells, but needs another protein to mediate the transforming actions of the cancer cell-derived MVs. We show that the tTG crosslinking substrate fibronectin (FN) also participates in transformation of non-cancerous cells. Specifically, and without intending to be constrained by any particular theory, we discovered that tTG crosslinks FN in MVs from cancer cells and that the ensuing MV-mediated transfer of crosslinked FN and tTG to recipient fibroblasts function cooperatively to activate mitogenic signaling activities and to induce their transformation. With respect to tTG crosslinking of FN, we demonstrate the presence of crosslinked FN in microvesicles shed from cancer cells, but we do not detect cross-linked FN in whole cell lysates (WCL) from the same cells (see, for example see FIG. 26). It is accordingly considered that FN crosslinking by tTG takes place in MVs. These findings highlight a novel role for MVs in the induction of cellular transformation. Accordingly, identifying MVs that have the capacity to induce cellular transformation, and providing methods for inhibiting this induction, are aspects of the present invention.

In connection with the present discoveries, there is a growing body of evidence demonstrating that cancer cells are capable of generating MVs in vivo. It has been shown that when MVs shed by cultures of several different human primary tumor cells or established cancer cell lines were subsequently added back to the same cancer cells, the growth and the survival of these cells were significantly enhanced. When considering these findings in the context of a tumor setting, the increased proliferative capacity afforded by sharing MVs among cancer cells could be envisioned as a mechanism to augment tumor growth. However, the present invention demonstrates that MVs impact cancer progression in an unexpected way, specifically, by conferring upon normal cell lineages that are major constituents of the tumor microenvironment (i.e. fibroblasts and epithelial cells), the transformed characteristics of cancer cells. This is consistent with the notion that the expansion of a tumor mass would not necessarily depend solely on the proliferation of the cancer cells, but rather could also include the aberrant growth exhibited by stromal cells (including fibroblasts) and normal epithelium in the tumor microenvironment that have been exposed to MVs shed by cancer cells.

In order for MVs shed from cancer cells (i.e., the MDAMB231 breast cancer cells and U87 brain tumor cells we have used as model cells) to promote the growth of normal cells (i.e. NIH3T3 fibroblasts and MCF10A mammary epithelial cells) in low serum and to induce their ability to form colonies in soft agar, the recipient cells in general need to be repeatedly treated with freshly prepared MVs during growth assays. This implies that the proteins and RNA transcripts contained within MVs that are involved in promoting their transforming activity, after being added to normal recipient cells, have a finite lifespan and need to be continuously replenished. When considering this in the context of a tumor setting, the chronic shedding of MVs by the cancer cells into their microenvironment might provide the continuous supply of MVs required by the nearby recipient stroma and normal epithelium to induce and maintain a transformed phenotype. Thus, the invention provides methods for detecting MVs involved in transformation and maintenance of the transformed phenotype conferred to non-cancer cells, and provides methods for inhibiting these processes.

In one embodiment, the present invention provides a method for characterizing microvesicles. The method in general comprises obtaining a sample and assaying the sample for the presence or absence of tTG associated with MVs. If tTG associated with microvesicles is present in the sample, the microvesicles are identified as tTG positive. If tTG is not identified in the sample, but microvesicles are nevertheless present, the microvesicles are identified as tTG negative. By “associated with” it is meant that the protein (i.e., tTG or FN) is present in a microvesicle, which includes being fully or partially contained within the microvesicle, or fully or partially within the vesicle membrane. However, in general, it is considered that tTG associated with MVs as described herein has at least a portion of the tTG protein as spanning the membrane of the MV or is associated with the outer leaflet of the MV membrane or outside the MV.

In other embodiments, the method comprises assaying microvesicles for cross-linked FN. Based on such assaying, the MVs can be identified as cross-linked FN positive if cross-linked FN associated with the microvesicles is detected. Likewise, the microvesicles are identified as cross-linked FN negative if cross-linked FN associated with the microvesicles is absent. Those skilled in the art will readily recognize how to differentiate cross-linked FN from non-cross-linked (monomeric) FN based on such factors as molecular weight, mobility analysis, etc.

In certain embodiments of the invention, determining that MVs are tTG positive can be indicative that the MVs are also cross-linked FN positive. Likewise, in certain embodiments, determining that the MVs are tTG negative can be indicative that the MVs are also cross-linked FN negative. It follows that in some embodiments, determining that MVs are cross-linked FN positive can be indicative that the MVs are tTG positive, and determining that the MVs are cross-linked FN negative can be indicative that the MVs are also tTG negative.

A related aspect of the invention provides a method which comprises detecting the presence of tTG positive and/or cross-linked FN positive microvesicles. The method comprises obtaining a sample and assaying the sample to detect the presence of microvesicles that are associated with tTG and/or with cross-linked FN.

In another aspect, the invention provides a method of diagnosing an individual as having circulating tTG positive and/or cross-linked FN positive microvesicles. This embodiment comprises assaying a sample obtained from the individual for tTG associated and/or cross-linked FN associated microvesicles, and identifying the individual as having circulating tTG associated microvesicles if tTG is present in the sample, or identifying the individual as having cross-linked FN positive MVs if cross-linked FN is present in the sample. The individual can be identified as not having circulating tTG associated microvesicles if tTG is absent from the sample, and can likewise be identified as not having circulating cross-linked FN associated MVs if cross-linked FN is absent from the sample.

As used herein, the term “microvesicles” or “MVs” is used to designate vesicles shed from cells, wherein the vesicles have a diameter ranging from 0.1 to 5.0 microns, inclusive, and including all integers there between and numbers to the tenth decimal point. Microvesicles that have the capacity to confer onto normal cells a transformed phenotype are also referred to herein as microvesicles or “oncosomes.” Some methods for detecting microvesicles shed from tumors are known in the art. See for example, (Skog J, et al. (2008) Nat Cell Biol 10:1470-1476) which discloses that brain tumor derived microvesicles could be detected in blood samples taken from human patients afflicted with glioblastoma microforme. Microvesicles as referred to in the present invention can also be identified if desired via the microvesicle surface marker CD63, or flotillin (see, for example, Rak 2008 Nature Cell Biology), or combinations of these and/or other MV markers.

In another aspect, the invention comprises a method for isolating membranous structures, which can include MVs, from a sample. The method generally comprises providing a sample which may comprise membranous structures, mixing the sample with tTG or a derivative thereof, and if the membranous structures are present in the sample, allowing formation of a complex of the membranous structures and tTG or a derivative thereof, and separating the complex of the tTG or tTG derivative and the membranous structures from the sample.

The membranous structures in various embodiments are generally spherical lipid containing bodies. The spherical membranous structure can comprise lipid bilayers. The method is particularly suited for capturing those membranous structures that are shed or otherwise secreted from cells. Thus, the membranous structures can be derived from any membrane containing biological material, which includes but is not necessarily limited to internal cellular membranes, vesicles, such as secretory vesicles, organelles, enveloped structures, plasma membranes and the like. In certain embodiments, the membranous structure is selected from vesicles, exosomes, microvesicles, micro-particles, intraluminal vesicles, endosomal derived vesicles, multivesicular bodies, and combinations thereof.

The invention is suitable for analyzing any biological sample for the presence of MVs, and in particular for tTG and/or cross-linked FN associated with microvesicles. In one embodiment, the sample is a liquid biological sample. The liquid biological sample can comprise or consist of blood, serum, cerebrospinal fluid, urine or any other biological fluid which can contain MVs. The biological sample can be obtained from an individual, such as a mammal. In one embodiment, the mammal is a human. The human can be an individual who has been diagnosed with, is at risk for developing, or has cancer. The biological sample can be used directly in determining the presence or absence of tTG and/or cross-linked FN positive microvesicles. In another embodiment, the sample is subjected to a processing step before the sample is tested. In some examples, the processing step can be carried out to purify microvesicles and/or enrich a sample for microvesicle content.

One aspect of the invention comprises separating microvesicles from a sample so that tTG positive MVs and/or cross-linked FN positive MVs in the sample, if present, can be identified. In various embodiments, separation of microvesicles or other membranous structures can be performed using any suitable technique or combination of techniques, which include but are not necessarily limited to approaches that separate compositions of matter based on size, density and/or charge, including but not limited to centrifugation and/or size exclusion chromatography methods. In various embodiments, microvesicles or other membranous structures can be detected and/or separated from a sample using one or more binding partners. Thus, the binding partner is an agent that can be used to detect, capture and/or separate microvesicles or other membranous structures from a sample. Separated microvesicles or other membranous structures can be tested to determine whether or not they comprise tTG and/or cross-linked FN, or other components, such as any peptide, protein, polynucleotide or any other marker that is informative of the origin of the microvesicle or other membranous structure, and/or its significance in assessing any disease or other condition. In various aspects of the invention determination of the presence or absence of tTG and/or cross-linked FN associated microvesicles or other membranous structures in a sample can be used in assessments of the status of the individual from which the sample was obtained with respect to one or more cancer related parameters, such as whether or not the individual has cancer, such as a solid tumor, and whether or not the individual has or is at risk for metastasis, and/or whether or not a particular treatment regime is providing a benefit to the individual from whom the sample comprising microvesicles was obtained.

In one embodiment, the binding partner used to separate microvesicles from a sample is an agent that can bind with specificity to tTG or to FN. Such agents include but are not necessarily limited to antibodies to tTG or FN, aptamers, and small molecule binders that can bind with specificity to tTG or crosslinked FN. In certain embodiments, the binding agent can bind with specificity to tTG or crosslinked FN that is associated with microvesicles that are shed from cancer cells. The binding agent can be specific for cross-linked FN that is associated with microvesicles. In another embodiment, the binding agent is specific for a complex of tTG and FN. Such complexes can be associated with microvesicles, and the binding agent can be specific for the microvesicle associated form.

In certain aspects of the invention, one or more binding partners can be used in immunoabsorbent-based detection, separation, and/or measurement of tTG positive and/or FN positive microvesicles. The binding partner can thus comprise or consist of anti-tTG antibodies or anti-FN antibodies or fragments of antibodies that can bind FN or tTG. The antibodies need not be of any particular class, and can be polyclonal or monoclonal antibodies. Antigen binding fragments include but are not necessarily limited to Fab, Fab′, (Fab′)₂, Fv, single chain (ScFv), diabodies, multi-valent antibodies, fusion proteins comprising one or more antibody portions, and any other modified immunoglobulin molecule that comprises an antigen recognition site of desired specificity for tTG, or for FN, or for a combination thereof.

With respect to immunoabsorbent-based detection of MVs, currently available isolation protocols that are known in the art generally involve a three-step centrifugation procedure that requires a large amount of starting material (i.e. conditioned medium from cancer cells or blood samples), is time consuming, and the resulting MVs yields can vary greatly from preparation to preparation. Thus, novel approaches to more efficient isolation of MVs and other membranous structures according to the invention have significant utility. Based on our findings showing that tTG and cross-linked fibronectin are components of cancer cell-derived MVs that have important consequences for their transforming capabilities as described more fully below, we developed tests to establish that we could capture MVs from conditioned medium from cancer cells using MV binding partners. In particular, we performed immunoprecipitations with a tTG antibody or a fibronectin antibody that had been pre-bound to protein G-agarose beads (purchased from Invitrogen) on conditioned medium collected from serum-starved MDAMB231 breast cancer cells and U87 brain tumor cells. As shown in FIG. 50, we demonstrate that MVs can be immunoprecipitated with either of these antibodies, as indicated by the presence of the MV marker flotillin in the immunoprecipitation lanes (third panel from the top). We also tested for the presence of RhoA, which is a protein that is known not to be a component of MVs, and it cannot be detected in these immunoprecipitations, which indicates that the medium is not contaminated with intact cells (bottom panel). Thus, we demonstrate capture of MVs shed from cancer cells using two different MV binding partners.

We also tested whether tTG itself could be used to capture MVs shed from cancer cells. To do this, we generated His-tagged recombinant tTG using conventional recombinant protein synthesis methods in bacteria. We associated this His-tagged recombinant tTG to nickel beads and tested whether recombinant tTG could also be used to isolate MVs from the conditioned medium collected from the cancer cell cultures. In this regard, the last two lanes in FIG. 50 show that this is indeed the case, as indicated by the detection of flotillin (third panel from the top) in these lanes. Thus, the data presented in FIG. 50 establish that a purified recombinant form of tTG can be used as an agent to capture MVs that are shed from cancer cells. The data presented in FIG. 50 also demonstrate that antibodies against tTG or fibronectin can be used as specific binding partners to isolate MVs that are shed by cancer cells.

As will be evident to the skilled artisan at least from in FIG. 50, certain aspects of the invention provide for use of recombinant tTG or a derivative thereof to capture MVs or other membranous structures from a sample. The amino acid sequence of human tTG is:

| (SEQ ID NO: 1) | |
|---|---|
| 1 | maeelvlerc dleletngrd hhtadlcrek lvvrrgqpfw ltlhfegrny easvdsltfs |
| 61 | vvtgpapsqe agtkarfplr daveegdwta tvvdqqdctl slqlttpana piglyrlsle |
| 121 | astgyqgssf vlghfillfn awcpadavyl dseeerqeyv ltqqgfiyqg sakfiknipw |
| 181 | nfgqfedgil diclilldvn pkflknagrd csrrsspvyv grvvsgmvnc nddqgvllgr |
| 241 | wdnnygdgvs pmswigsvdi lrrwknhgcq rvkygqcwvf aavactvlrc lgiptrvvtn |
| 301 | ynsandqnsn llieyfrnef geiqgdksem iwnfhcwves wmtrpdlqpg yegwqaldpt |
| 361 | pqeksegtyc cgpvpvraik egdlstkyda pfvfaevnad vvdwiqqddg svhksinrsl |
| 421 | ivglkistks vgrderedit htykypegss eereaftran hlnklaekee tgmamrirvg |
| 481 | qsmnmgsdfd vfahitnnta eeyvcrlllc artvsyngil gpecgtkyll nlnlepfsek |
| 541 | svplcilyek yrdcltesnl ikvrallvep vinsyllaer dlylenpeik irilgepkqk |
| 601 | rklvaevslq nplpvalegc tftvegaglt eeqktveipd pveageevkv rmdllplhmg |
| 661 | lhklvvnfes dklkavkgfr nviigpa. |

The invention includes using full length recombinantly produced tTG as a binding agent.

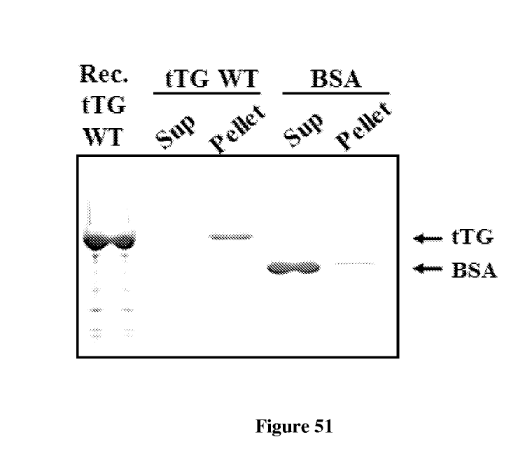

The invention also includes using tTG derivatives. tTG derivatives suitable for use in the invention include but are not necessarily limited to deletions, insertions, and conservative amino acid substitutions to the sequence of SEQ ID NO:1. Those skilled in the art will recognize that various modifications can be made to the sequence without affecting its utility for capturing MVs and other membranous structures. For example, the tTG sequence can be modified to include amino acid based tags that facilitate protein purification, such as a His-tag. Also included are mutated forms of tTG that have modified enzymatic activity. For instance, in one embodiment, a mutated tTG used in the invention is a transamidation (cross-linking) deficient tTG Amino acid sequences of transamidation deficient tTG proteins are known in the art. In one embodiment, the transamidation deficient tTG comprises a mutation selected from a change of cysteine 277 to valine (C277V), a change of cysteine 277 to serine (C277S), a double mutant where aspartic acid 306 and asparagine 310 are changed to alanines (D306A/N310A, which is also referred to as the site 2 mutant), and combinations of these mutations. In another embodiment, the tTG derivative is a fibronectin-deficient-binding form of tTG. Such tTG derivatives comprise the sequence of SEQ ID NO:1, but lack the first seven amino acids of SEQ ID NO:1. In other embodiments, the tTG derivative used in the invention comprises or consists of amino acid residues 1-139 (the so-called N-terminal β-sandwich domain) or amino acids 1-200 of tTG. In this regard, we have demonstrated that these truncated forms of tTG, when expressed in cells, have the ability to strongly associate with plasma membranes (i.e., see FIG. 51 and description of it below). Thus, it is expected that these tTG derivatives will be able to capture MVs and other membranous structures from a sample in accordance with the invention.

Any suitable tTG or FN binding partner or tTG or a derivative thereof, or combination of the foregoing, can be used to determine whether or not a sample comprises microvesicles that are associated with tTG. In certain aspects of the invention, only one type of binding partner is used. As an alternative to or in addition to a tTG and/or FN specific binding partner, a binding partner that can bind to a microvesicle marker other than tTG or FN can be used. For instance, antibodies or antigen binding fragments thereof which specifically recognize microvesicle markers other than tTG or FN can be used to capture microvesicles, irrespective of whether or not the captured microvesicles are associated with tTG. A binding partner that recognizes a microvesicle marker that is not tTG or FN can be considered a general microvesicle binding partner. In various embodiments, the general MV binding partner can be CD63 or flotillin. The general microvesicle binding partner can be used to obtain a first population of microvesicles. The first population of microvesicles can be homogeneous for microvesicles which are not associated with tTG, or homogeneous for microvesicles which are associated with tTG, or it can comprise a mixed population of microvesicles, some of which are associated with tTG and some of which are not. The first population of microvesicles can be assayed for the presence or absence of tTG using any suitable technique. The presence of tTG is indicative that the first population of microvesicles comprises microvesicles that are associated with tTG. Likewise, the absence of tTG is indicative that the first population does not contain tTG associated microvesicles. If desired, a measurement of the amount of tTG associated microvesicles in the first population can be made. For example, an assessment of an amount of tTG (if present) in the first population can be made and used to assess the amount of tTG associated microvesicles in the first population. In another embodiment, a first population of microvesicles can be subjected to further separation using a tTG and/or FN specific binding partner to obtain a second population of microvesicles. Thus, a second population of tTG associated microvesicles can be obtained from a first population of microvesicles and can accordingly be enriched for microvesicles that contain tTG and/or FN. Alternatively, more than one sample can be processed using tTG and/or FN binding partner(s) to provide a composition that is enriched with microvesicles that are associated with tTG.

In one embodiment the invention provides a method of using tTG or a derivative of tTG to capture any membranous structures that are shed from cells. This is based in part on our demonstration that tTG can be used to capture MVs shed from cancer cells as described above. In connection with this aspect of the invention, we tested the capability of tTG to directly associate with plasma membranes, and whether it requires additional proteins to do so. The results of this analysis are presented in FIG. 51. To obtain these results, purified recombinant wild-type tTG (tTG WT), or bovine serum albumin (BSA) serving as a control, was combined together with synthetically-derived liposomes whose lipid composition was similar to that of the inner leaflet of the plasma membrane in mammalian cells. Following a brief incubation, the vesicles were pelleted by centrifugation, and then the resulting supernatants and pellets were resolved by SDS-PAGE and stained with Quick Blue to detect the proteins. FIG. 51 shows that tTG has a relatively high affinity for lipids, as nearly all of the recombinant tTG (tTG WT) was found to have pelleted with the synthetic vesicles. On the other hand, bovine serum albumin (BSA) only weakly pelleted with the liposomes, with most of the control protein remaining in the supernatant. Thus, we have demonstrated that tTG can be used to not only isolate MVs shed from cancer cells, but it can also be used to isolate synthetic membranous structures that are devoid of any cellular components. Thus, recombinant tTG constitutes in one embodiment of the invention a general membranous structure binding partner.

In various embodiments, the presence or absence of tTG and/or cross-linked FN in a sample can be detected using any of a variety of approaches for detecting proteins, such as immunodetection methods, including but not limited to Western blotting, multi-well assay plates adapted for detection of proteins, beads adapted for detection of proteins, a lateral flow device or strip that is adapted for detection of proteins, ELISA assays, or any other modification of an immunodetection or other assay type that is suitable for detecting proteins. Those skilled in the art will recognize that, given the benefit of the present disclosure, these and other detection methods can include use of one or more tTG or FN binding partners as described herein. In various embodiments, the one or more binding partners can be reversibly or irreversibly attached to a substrate, such as by being covalently, ionically, or physically bound to a solid-phase immunoabsorbent using methods such as covalent bonding via an amide or ester linkage, ionic attraction, or by adsorption. The substrate can be any suitable substrate onto which a binding partner can be attached. Examples include substrates typically used in immunodetection assays, lateral flow devices, bead-based assays and the like. The solid substrate can be a porous solid substrate that allows the flow of liquid through the substrate. The liquid can flow through the porous substrate via any suitable means, such as by capillary action, microfluidics, etc. The substrate can also be a non-porous solid substrate, such as beads formed from glass or other non-porous materials.

The invention includes use of a detection component, which can be used in various assays that are suitable for detecting the presence or absence of tTG and/or cross-linked FN associated with microvesicles according to the invention. The detection component can be, for example, any reagent that can be used to detect the presence of tTG and/or cross-linked FN when it is bound to a specific binding partner. In some embodiments, the detection component comprises a radioactive tag, a fluorescent tag, or a chemiluminescent tag or substrate. In some embodiments, the detection component can be part of a substrate to which a binding partner of the invention is attached. For example, one or more beads which comprise a detectable label, such as a fluorescent label, or a bar code, or another means by which the beads and the presence or absence of tTG and/or cross-linked FN can be ascertained can be used. Those skilled in the art will recognize that such configurations of reagents will permit for multiplexed assays.

The amount of tTG and/or cross-linked FN, and/or the amount of tTG positive MVs and/or cross-linked FN MVs determined by the method of the invention can be compared to a reference. The reference can be determined using any method known to those of ordinary skill in the art. In various embodiments, comparison to a reference can be performed to ascertain prognostic significance of the presence of and/or the amount of tTG cross-linked FN positive microvesicles. For example, the reference can be a reference level of tTG cross-linked FN or a reference level of tTG positive cross-linked FN positive microvesicles. The reference level may be a known value or range of values, or may be a value or range of values determined from, for instance, one or more subjects known to be free of cancer, or free of tTG positive microvesicles and/or cross-linked FN positive MVs, or one or more subjects with various types and/or stages of cancer. In some embodiments, the reference level is an average level determined from a cohort of subjects with cancer, such as a particular type of cancer and/or a particular stage of cancer. The reference may constitute a control, such as a control sample that is free of tTG and/or cross-linked FN or free of tTG cross-linked FN positive microvesicles, or is loaded with a known amount of tTG and/or cross-linked FN or tTG and/or cross-linked FN positive microvesicles.

It is expected that the present invention can be used in connection with a wide variety of neoplastic disorders. For example, tTG and cross-linked FN positive microvesicles are expected to be associated with a variety of solid tumors. Further, tTG and cross-linked FN positive microvesicles are expected to be indicative of metastasis, or a risk of metastasis, in the individual from whom the microvesicles are isolated.

In certain embodiments, the invention provides a method of diagnosing an individual as having circulating microvesicles that are tTG positive, or cross-linked FN positive, or tTG negative, or cross-linked FN negative. This comprises assaying a sample obtained from the individual for tTG and/or cross-linked FN negative associated with microvesicles, and identifying the individual has having circulating tTG and/or cross-linked FN associated microvesicles if tTG and/or cross-linked FN is present, and identifying the individual as not having circulating tTG and/or cross-linked FN associated microvesicles if tTG and/or cross-linked FN is absent from the sample. It is considered that the circulating microvesicles are those which travel through the blood or other bodily fluids of an individual. In one embodiment, the sample analyzed using the method of the individual is obtained from an individual who has been diagnosed with cancer and is undergoing cancer therapy.

There is no particular limit to the type of cancer types that the invention is expected to be useful for diagnosis, prognosis and development of recommended therapeutic interventions, so long as the cancer involves formation of tTG and/or cross-linked FN positive microvesicles. In this regard, examples of cancers with which the present invention is expected to be valuable include but are not limited to fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, and Waldenstrom's macroglobulinemia.

In another aspect the invention provides a method for inhibiting in an individual transfer of cargo from microvesicles which comprises tTG to one or more cells in the individual. Inhibition of transfer of cargo can include but is not necessarily limited to inhibiting tTG positive microvesicle from docking to a cell, and inhibiting some or all of the contents of a microvesicle from entering a cell to which a microvesicle may have fully or partially docked. This method comprises administering to the individual a composition comprising a tTG inhibitor. In one embodiment, the tTG inhibitor is a cell-impermeable inhibitor. In one embodiment, the composition is administered to an individual who has been diagnosed with, is suspected of having, or is at risk for cancer. In one embodiment, the administration of the composition results in an inhibition of metastasis and/or inhibition of formation of metastatic foci in the individual.

The individual to whom the composition comprising a cell-impermeable tTG inhibitor is administered can be an individual in need of tTG inhibition, and/or an individual who has been diagnosed with, is suspected of having, or is at risk for developing a disease or other disorder that is associated with tTG positive microvesicles, including but not necessarily limited to all cancers described herein. In one embodiment, the individual to whom the composition is administered does not have an autoimmune disease and/or has not previously undergone therapy for an autoimmune disease. In certain examples, the individual does not have, and/or has not and is not undergoing therapy for coeliac disease. In certain instances, the individual is an individual to whom an agent that can specifically inhibit tTG has not previously been administered.

The cell impermeable tTG inhibitor can be an inhibitor that can specifically inhibits the crosslinking activity of tTG or one which acts sterically, such as by interfering with tTG positive microvesicles docking to a cell by blocking tTG from interacting with, for example, a cell surface receptor. In one embodiment, the cell-impermeable tTG inhibitor is a biologic agent, such as an anti-tTG antibody, or a tTG binding fragment thereof. tTG binding fragments of antibodies are described above and are expected to be suitable for therapeutic purposes. The antibody or antigen binding fragment of it can be monoclonal in nature, or a recombinantly generated antibody or antigen binding fragment. These agents may be chimeric, partially humanized or fully humanized with respect to their amino acid content. In various embodiments, the inhibitor is an antibody or antigen binding fragment thereof that can specifically recognize tTG that is associated with a microvesicle. In another embodiment, the inhibitor is an antibody or antigen binding fragment thereof that can recognize a complex of tTG and crosslinked FN, which can in particular examples be associated with a microvesicle.

In another aspect, the tTG inhibitor is a small molecule that can specifically inhibit the crosslinking activity of tTG. In one embodiment, the tTG inhibitor is known in the art as T101. T101 is commercially available from Zedira (Darmstadt, Germany). Other tTGG inhibitors include but are not necessarily limited to the compound known as BOC-DON (B003) from Zedira, or the compound known as KCC 009, which is described by Yuan et al., Oncogene (2007) 26, 2563-2573.

The tTG inhibitors can be provided in compositions such as pharmaceutical preparations. Compositions for use in therapeutic purposes may be prepared by mixing the inhibitor with any suitable pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins. It will be recognized by those of skill in the art that the form and character of the particular dosing regimen for any tTG inhibitor employed in the method of the invention will be dictated by the route of administration and other well-known variables, taking into account such factors as the size, gender, health and age of the individual to be treated, and the type and stage of a disease with which the individual may be suspected of having or may have been diagnosed with. Based on such criteria, one skilled in the art can determine an effective amount of a composition to administer to the individual.

Compositions comprising tTG inhibitors can be administered to an individual using any available method and route, including oral, parenteral, subcutaneous, intraperitoneal, intrapulmonary, intranasal and intracranial injections. Parenteral infusions include intramuscular, intravenous, intraarterial, intraperitoneal, and subcutaneous administration. The method of the invention can be performed prior to, concurrently, or subsequent to conventional anti-cancer therapies, including but not limited to chemotherapies, surgical interventions, and radiation therapy.

In various embodiments, the invention comprises fixing in a tangible medium the determination of whether or not a sample containing contained tTG and/or cross-linked FN associated microvesicles. The tangible medium can be any type of tangible medium, such as any type of digital medium, including but not limited a DVD, a CD-ROM, a portable flash memory device, etc. The invention includes providing the tangible medium to a health care provider to develop a recommendation for treatment of an individual from which a tTG positive microvesicle sample was obtained.

In various embodiments, the present invention provides kits for determining whether a composition comprises microvesicles or other membranous structure that are associated with tTG and/or cross-linked FN. The kit can comprise reagents for capturing microvesicles or other membranous structure which comprise tTG and/or cross-linked FN, such as one or more tTG and/or cross-linked FN specific binding partners. The kits can also comprise recombinant tTG or a derivative thereof. The specific binding partner(s) and reagents for use in assaying microvesicles can be contained in one or more sealed, sterile vials. The kits can contain instructions for assaying microvesicles and/or samples comprising microvesicles for the presence of tTG and/or cross-linked FN. Thus, the kits can contain tools for immunodetection of tTG and/or cross-linked FN positive microvesicles, such as a lateral flow device, or beads that have been complexed with the one or more binding partners described herein.

In another aspect of the invention, a composition comprising an isolated population of microvesicles or other membranous structure, wherein the microvesicles or other membranous structure comprise tTG and/or cross-linked FN, and wherein the isolated population of microvesicles or other membranous structure is attached to tTG or a derivative thereof, or to a tTG and/or cross-linked FN binding partner. As an alternative or in addition to the microvesicles or other membranous structure being attached to tTG or a derivative thereof, and/or a tTG or cross-linked FN binding partner, compositions of the invention can also comprise an isolated population of microvesicles, wherein the microvesicles comprise tTG and cross-linked FN, and wherein the isolated population of microvesicles is attached to a FN binding partner. The tTG binding partner and/or the FN binding partner in various embodiments of such compositions can be attached to a solid substrate as further described above.

The following examples are presented to illustrate the present invention. They are not intended to limiting in any manner.

### Example 1

This Example presents results obtained by performing experiments using the materials and methods described in Example 3.

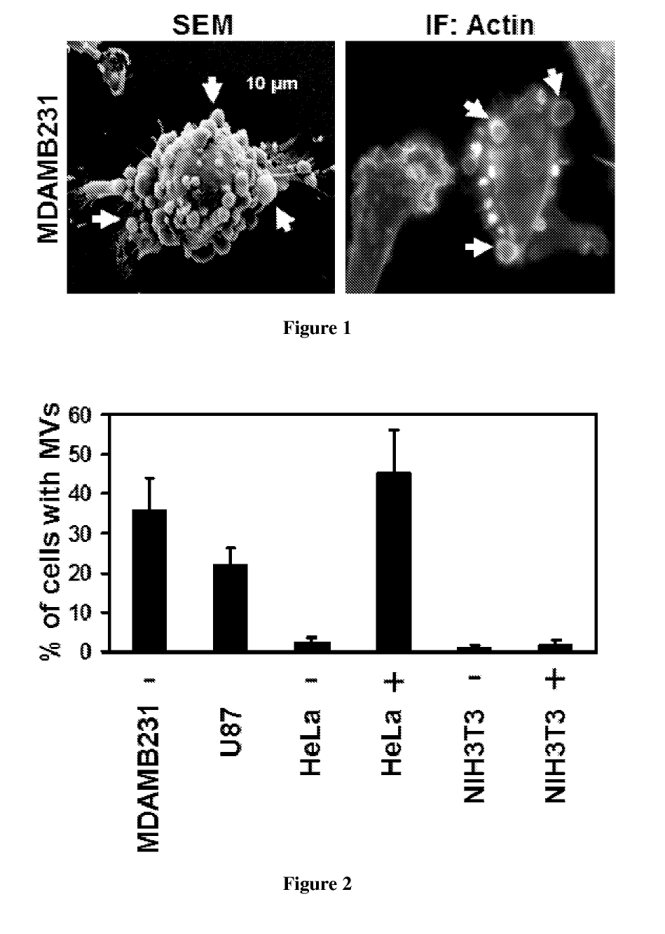

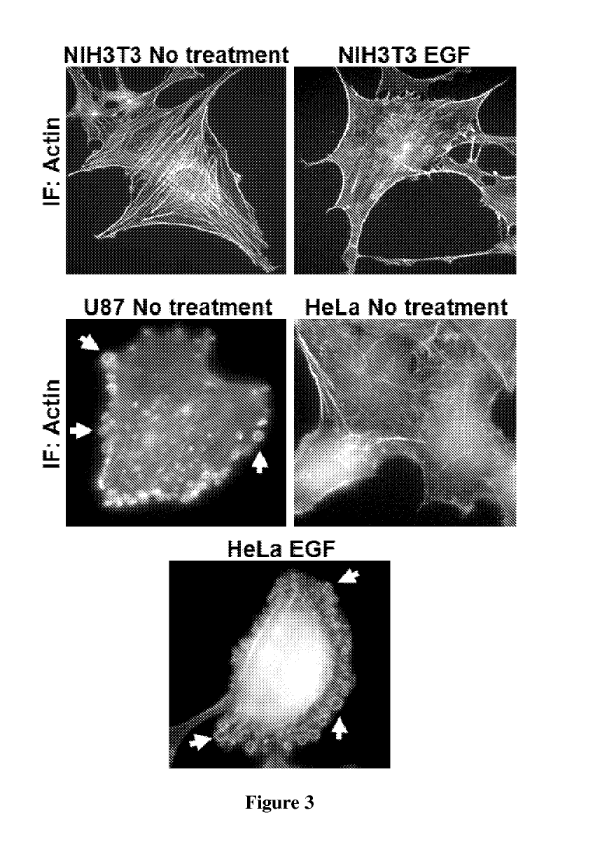

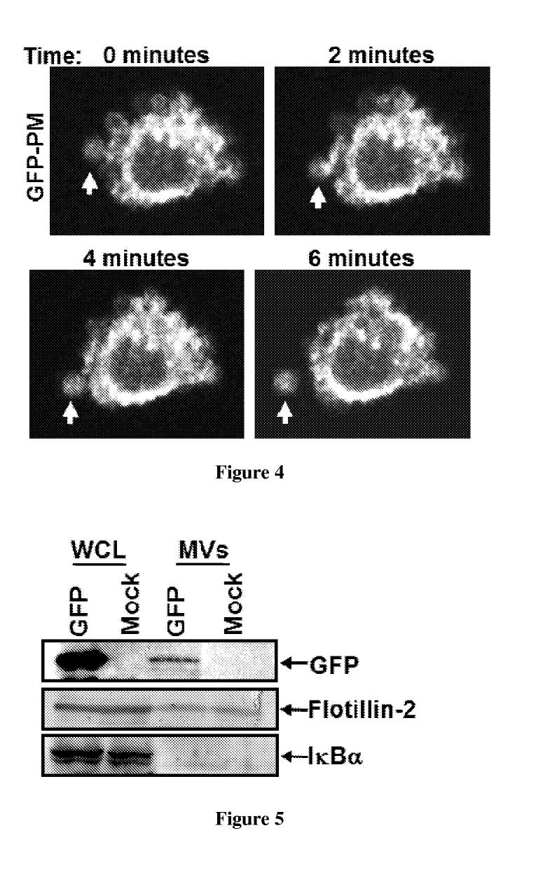

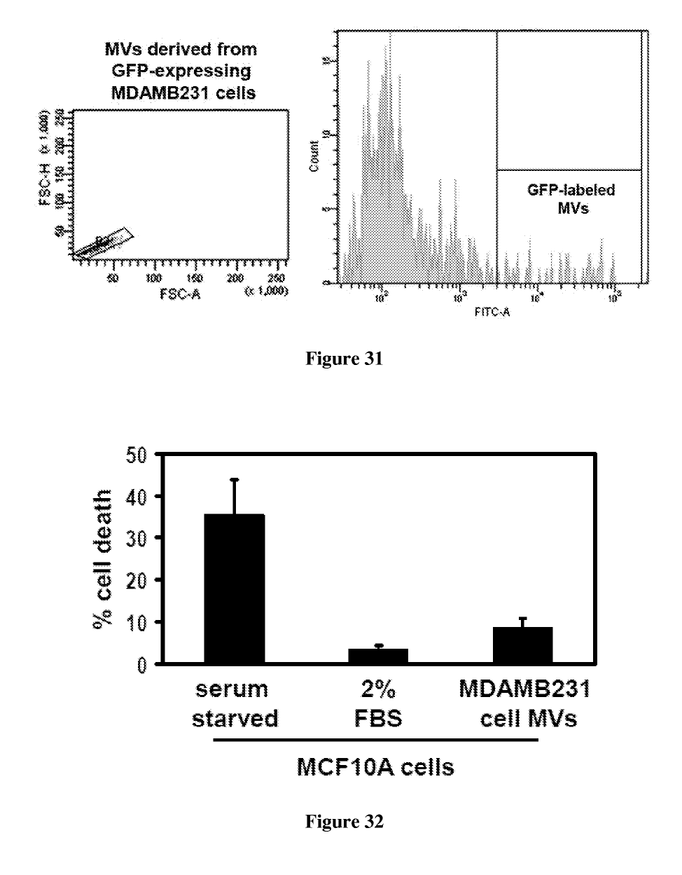

We analyzed serum-starved cultures of the highly aggressive human breast cancer cell line MDAMB231 by scanning electron microscopy (SEM) (FIG. 1, left image) or by fluorescent microscopy performed on cells stained for F-actin (FIG. 1, right image) and showed that MVs ranging in size from ˜0.2-2.0 microns in diameter were present on the surface of ˜35% of these cells (FIG. 2.). MVs were also detected on ˜25% of serum-deprived U87 human glioma cells, while their formation was induced in HeLa cervical carcinoma cells by epidermal growth factor (EGF) stimulation (FIGS. 2 and 3). In contrast, MVs were not detected on the surface of normal NIH3T3 fibroblasts cultured under serum-starved or EGF-stimulated conditions, indicating that some cell types may not generate MVs. Moreover, it was determined that MVs were actively shed from these cancer cells as demonstrated by time-lapsed images of the release of a GFP-labeled MV from the plasma membrane of a MDAMB231 cell transfected with a pEGFP plasmid encoding the plasma membrane targeting sequence of the Lyn tyrosine kinase (GFP-PM) (FIG. 4), as well as through the detection of MVs containing GFP in the culturing medium collected from pEGFP-only expressing transfectants by immunoblot analysis (FIG. 5) and fluorescence-activated cell sorting (FACS) analysis (FIGS. 30 and 31).

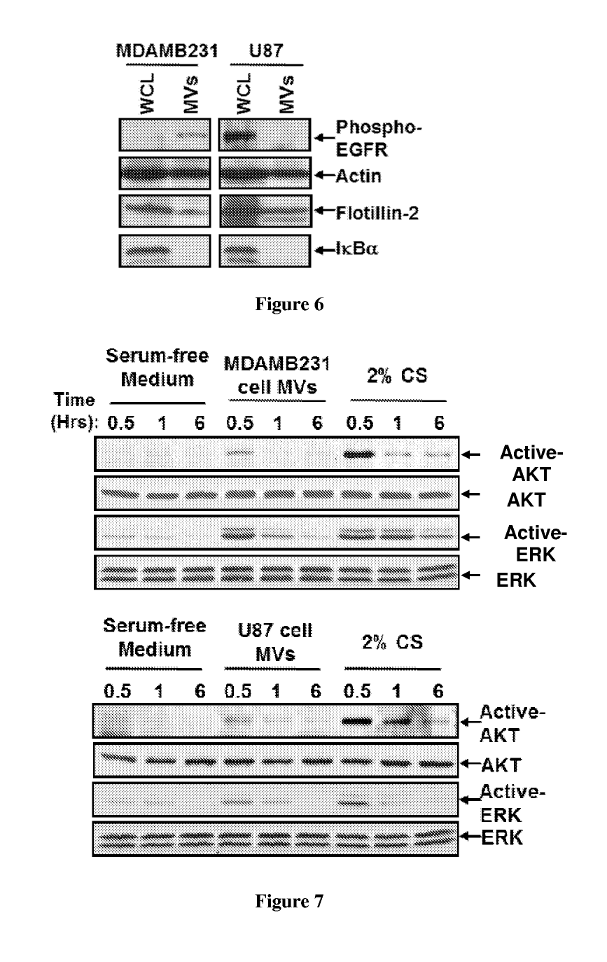

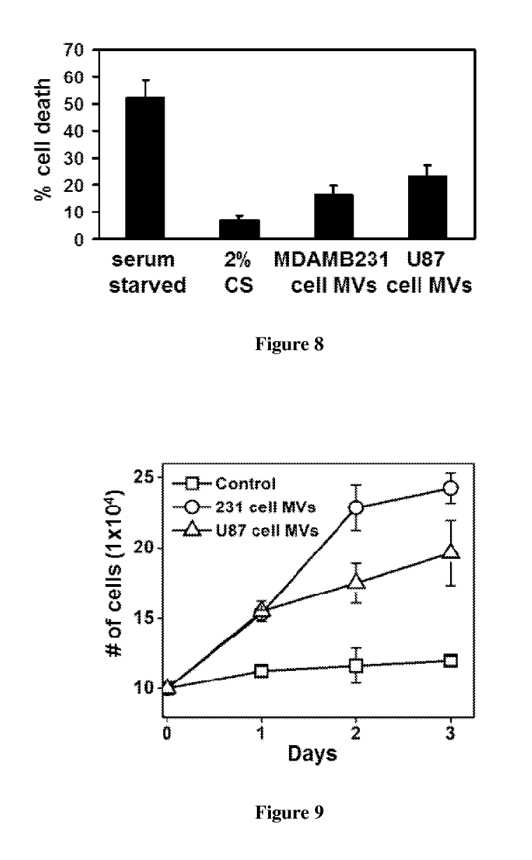

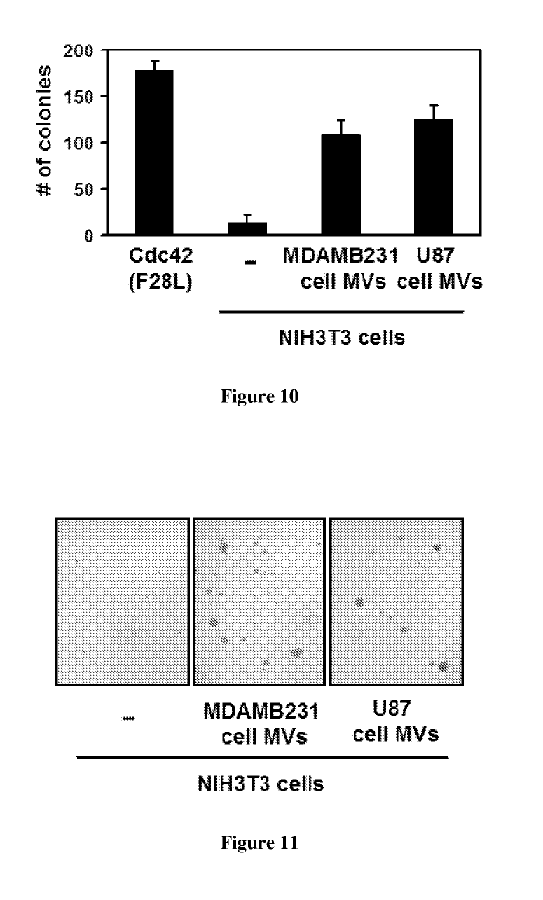

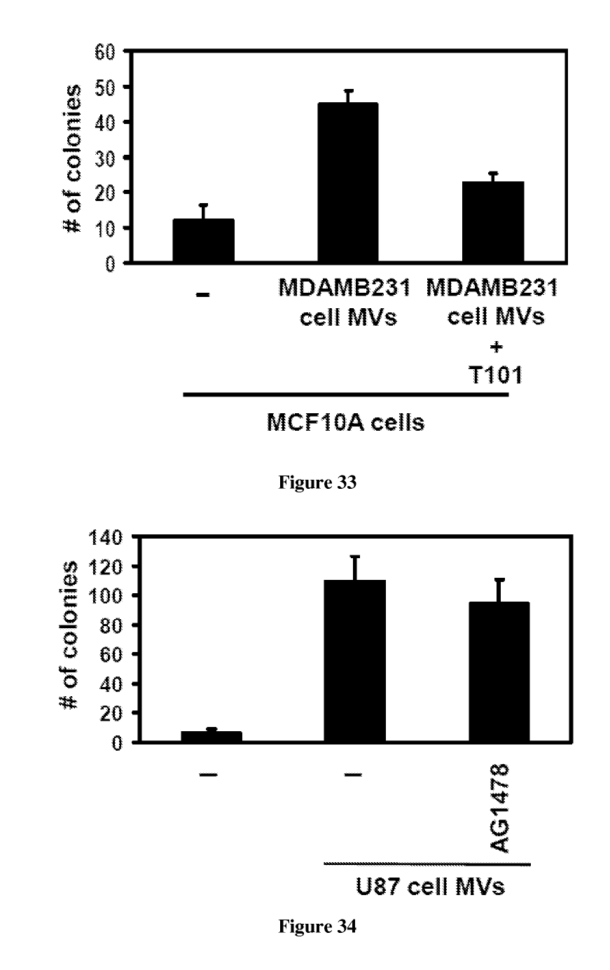

While MVs have been previously reported to share their cargo between cancer cells, we tested whether MVs might be capable of conferring onto normal (non-transformed) recipient cells some of the transformed characteristics of the donor cancer cells. Thus, we took isolated MVs constitutively shed by MDAMB231 breast cancer cells and U87 brain tumor cells from their serum-free culturing medium (FIG. 6) and added them to cultures of non-transformed NIH3T3 fibroblasts. The MVs generated by either of these cancer cell lines were capable of stimulating the activities of the signaling protein kinases AKT and ERK in the recipient fibroblasts (FIG. 7), similar to what has been observed when cancer cell-derived MVs were incubated with other cancer cells or endothelial cells. Moreover, when NIH3T3 fibroblasts were incubated with MVs derived from MDAMB231 cells or U87 cells, they exhibited two phenotypes characteristic of cancer cells, namely an enhanced survival capability (FIG. 8) and an ability to grow under low serum conditions (FIG. 9). We then tested whether the cancer cell-derived MVs, when added to normal cells, could induce cellular transformation as read-out by anchorage-independent growth (i.e. colony formation in soft agar). FIGS. 10 and 11 show that while the control NIH3T3 fibroblasts failed to form colonies in soft agar, sustained treatment of fibroblasts with MVs collected from either MDAMB231 cells or U87 cells conferred upon them the ability to grow under anchorage-independent conditions. MDAMB231 cell-derived MVs similarly promoted the survival (FIG. 32) and aberrant growth (FIG. 33) of the normal human mammary epithelial cell line MCF10A. Thus, the continuous MV-mediated transfer of cargo from cancer cells to normal cells is indeed capable of endowing these cells with the characteristics induced by oncogenic transformation.

We then analyzed what MV-associated protein(s) is responsible for mediating the transfer of transforming capability. Initially considered was the EGF-receptor as a possible candidate protein, since it was shown that activated forms of this receptor can be shared between brain cancer cells via MVs. However, it is unlikely that the EGF-receptor accounts for the similar transforming abilities associated with the MVs derived from MDAMB231 breast cancer cells and U87 glioblastoma cells (FIGS. 8-11), given that activated EGF-receptors cannot be detected in the MVs shed from U87 cells (FIG. 6). This was further supported by the finding that the anchorage-independent growth advantage imparted to NIH3T3 cells by U87 cell-derived MVs is insensitive to treatment with the EGF-receptor tyrosine kinase inhibitor AG1478 (FIG. 34).

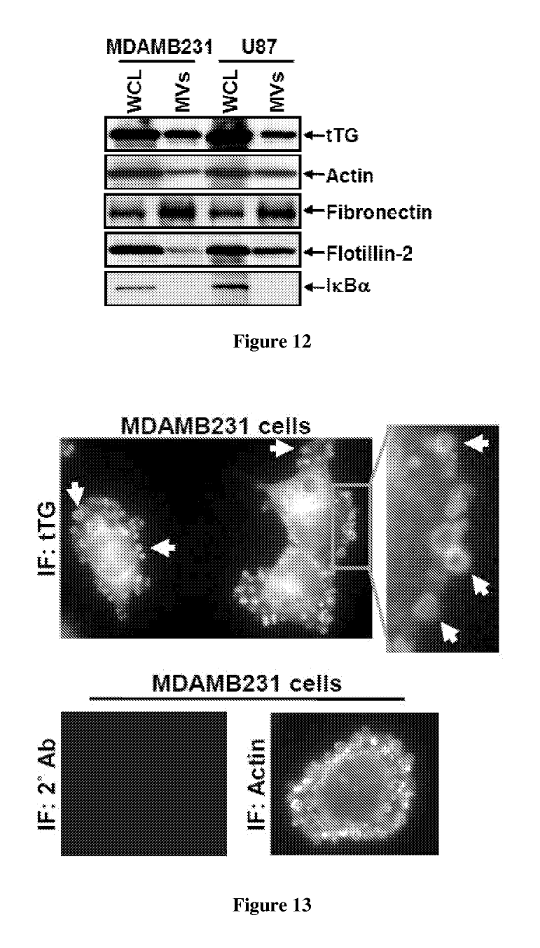

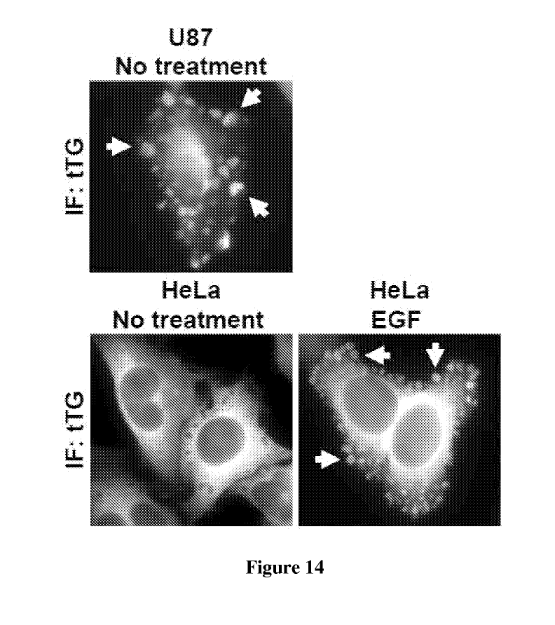

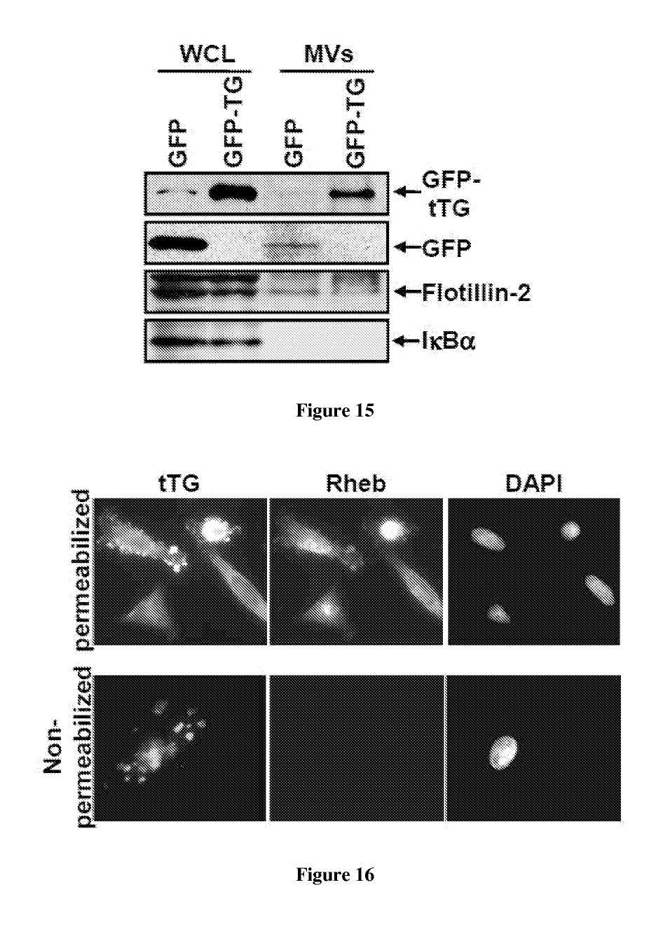

To identify proteins potentially involved in the transforming actions of these MVs, proteomic screens were carried-out. Proteins common to MVs derived from MDAMB231 cells and U87 cells are listed in Example 3. Notably among the MV-associated proteins was tTG, a protein crosslinking enzyme that has been linked to the chemoresistance and aberrant cell growth exhibited by some cancer cells, and is secreted from cells by an unknown mechanism. It was determined that tTG is a component of MVs derived from MDAMB231 and U87 cells by immunoblot analysis (FIG. 12) and demonstrated that the MVs on the surfaces of MDAMB231 cells were detectable when immunostained with a tTG antibody (FIG. 13, top images), but not when stained with only the secondary antibody (FIG. 13, bottom-left image). Likewise, the MVs generated by U87 cells and HeLa cervical carcinoma cells stimulated with EGF also contained tTG (FIG. 14). These findings, when coupled with the fact that a GFP-tagged form of tTG is more efficiently incorporated into MVs shed by MDAMB231 compared to GFP alone (FIG. 15), demonstrate that tTG is targeted to MVs generated by distinct types of cancer cells and in response to specific cell culturing conditions.

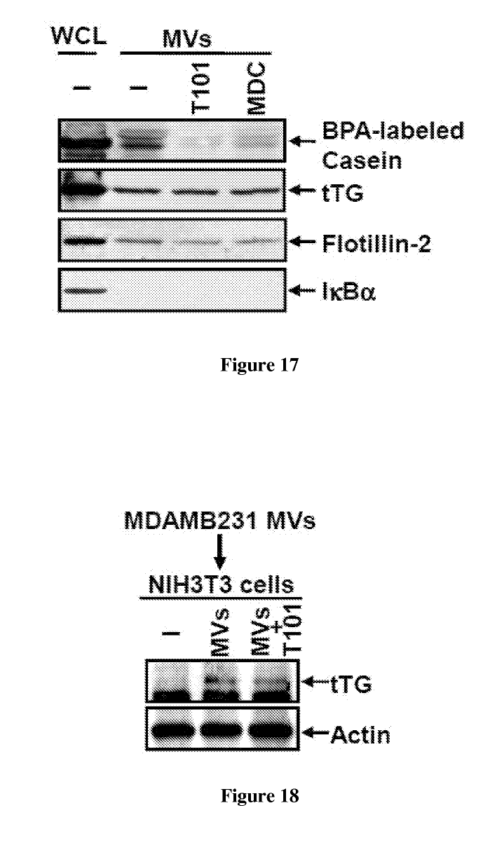

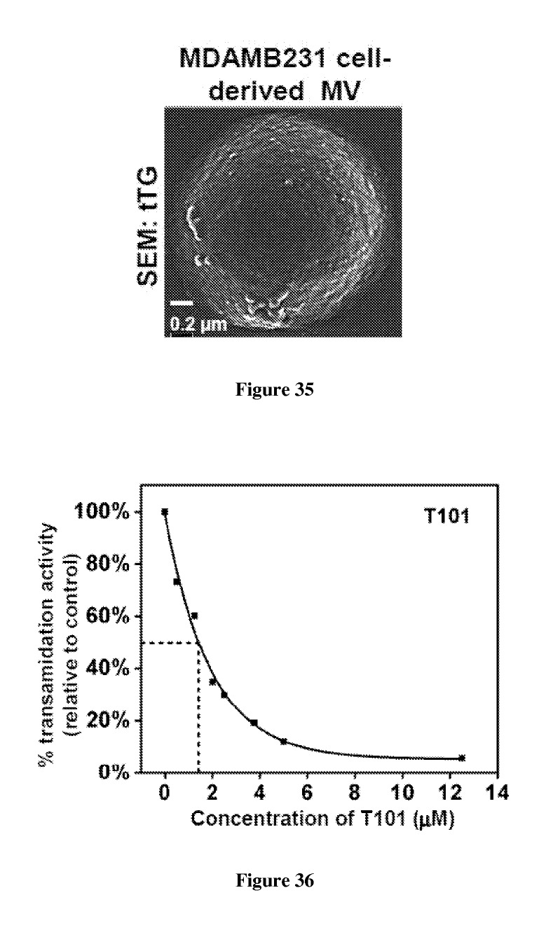

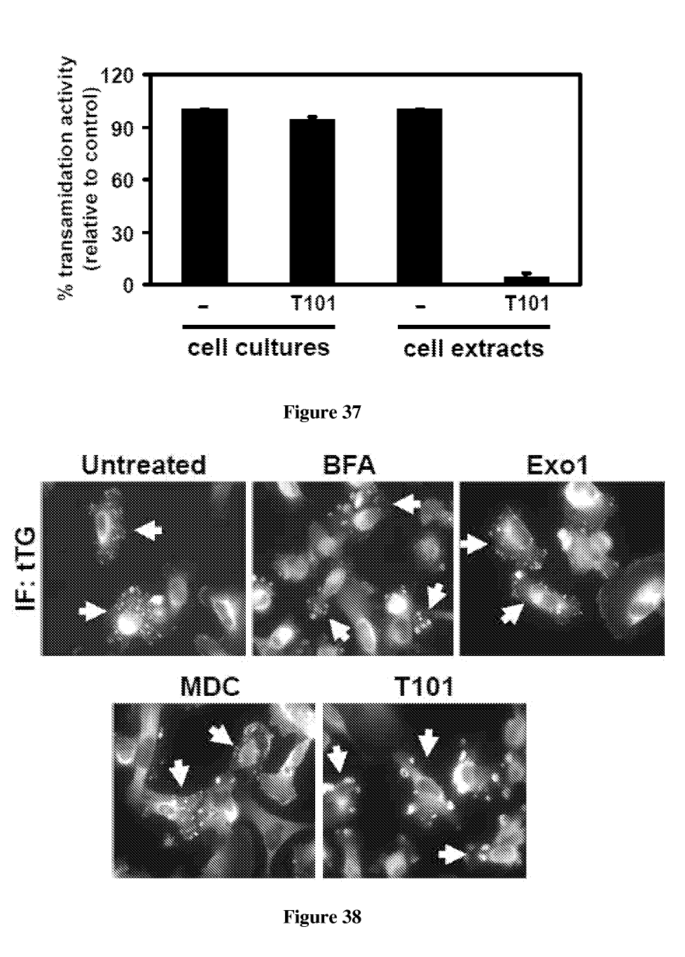

As shown in FIG. 13, tTG was frequently enriched in the membranes of MVs, as indicated by the ring-shaped staining patterns detected with a tTG antibody in cells actively forming MVs. The same tTG antibody also labeled MVs that protruded from the plasma membranes of non-permeabilized MDAMB231 cells (FIG. 16), as well as detected tTG on the surfaces of individually isolated MVs from MDAMB231 cells by immuno-SEM (FIG. 35). The top panel in FIG. 17 shows that tTG expressed in whole cell lysates (WCL) from MDAMB231 cells, or in intact MVs shed by these cells, was enzymatically active as readout by its ability to catalyze the incorporation of biotinylated pentylamine (BPA) into casein. Pre-treatment of the intact MDAMB231 cell-derived MVs with the cell permeable tTG inhibitor monodansylcadaverine (MDC) greatly diminished the levels of BPA-labeled casein detected in the assay. Interestingly, the cell impermeable tTG inhibitor T101 (FIGS. 36 and 37) also effectively blocked the crosslinking activity associated with MVs derived from MDAMB231 cells (FIG. 17), suggesting that tTG is predominantly localized and activated on the outer leaflet of MV membranes.

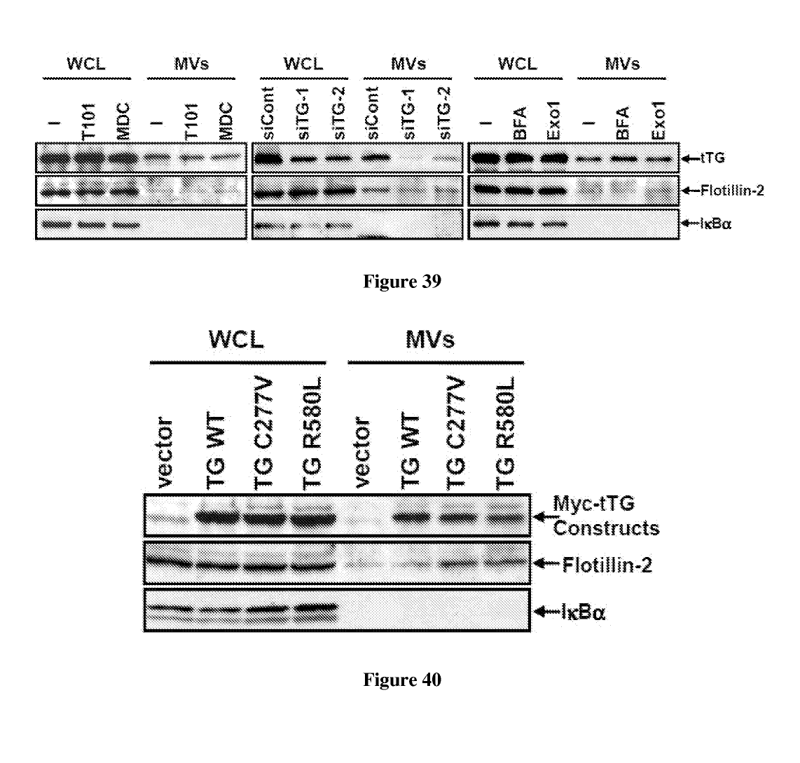

The MVs derived from MDAMB231 breast cancer cells were not sensitive to the traditional secretory inhibitors, BFA or ExoI, which block Arf GTPase activation, as indicated by monitoring MV formation by immunofluorescence staining of vesicle-associated tTG (FIG. 38). Next we tested the possibility that the ability of tTG to crosslink proteins was important for the formation and/or shedding of MVs by cancer cells Immunofluorescent analysis with a tTG antibody revealed that exposing MDAMB231 cells to the tTG inhibitors MDC and T101 had no effect on MV formation (FIG. 38). The shedding of MVs by MDAMB231 cells also did not require tTG enzymatic activity, nor was it affected by ExoI or BFA, as shown by the detection of nearly equivalents amounts of the MV-marker flotillin-2 and tTG in MVs isolated from the culturing medium of control cells or cells treated with different inhibitors (FIG. 39, left and right panels). Correspondingly, knocking-down tTG in MDAMB231 cells, which depleted the expression of tTG in the MVs, caused little change in the amount of MVs shed by these cells as read-out by the flotillin-2 marker (FIG. 39, middle panel). Moreover, tTG mutants, defective in their ability to crosslink substrates (tTG C277V) or to bind GTP (tTG R580L), when ectopically expressed in MDAMB231 cells were targeted to MVs as efficiently as ectopically expressed, wild-type tTG (FIG. 40). Thus, these results indicate that tTG is not essential for the ability of cancer cells to form or shed MVs, nor is the enzymatic activity of tTG needed for its targeting to MVs.

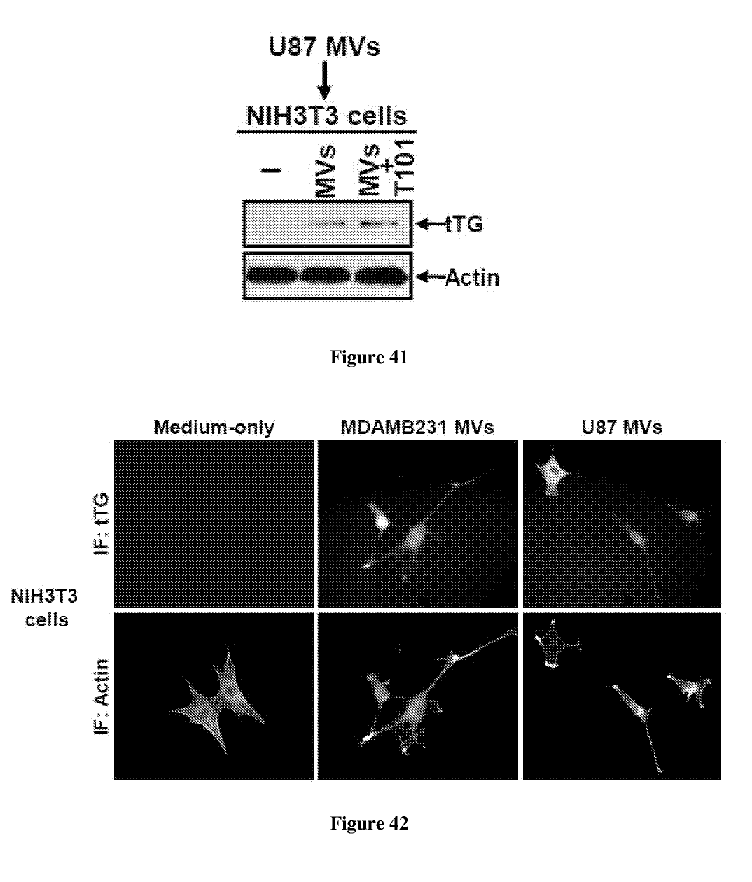

Next examined was whether tTG might function as MV cargo and be transferred to recipient cells. NIH3T3 fibroblasts were incubated for 30 minutes with MVs derived from serum-starved cultures of either MDAMB231 cells or U87 cells and then analyzed for tTG expression by immunoblot analysis (FIGS. 18 and 41) and immunofluorescent microscopy (FIG. 42). The results from these experiments show that the levels of tTG were significantly increased in fibroblasts that had been incubated with the cancer cell-derived MVs relative to the barely discernible levels of tTG in control fibroblasts.

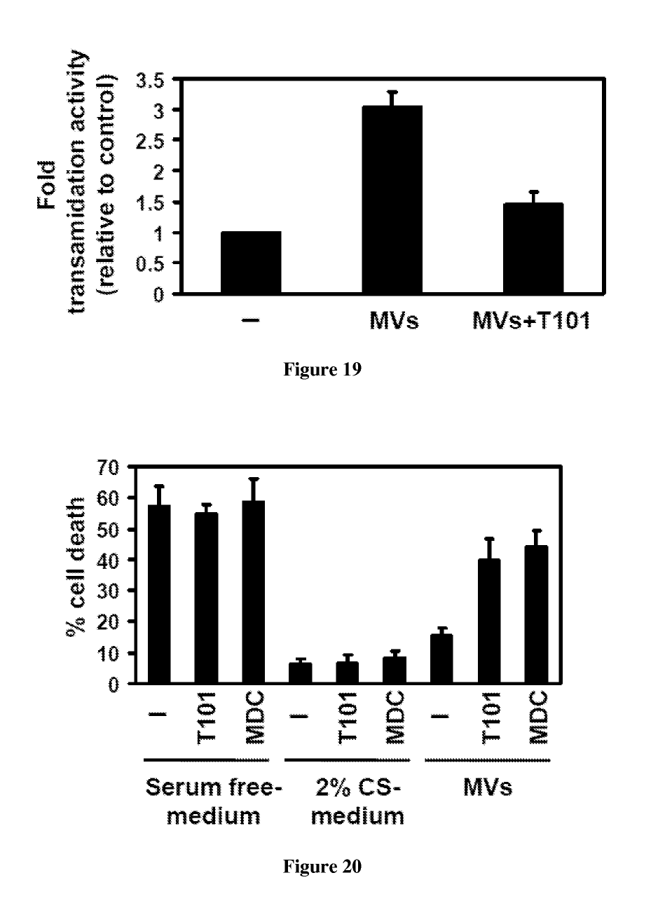

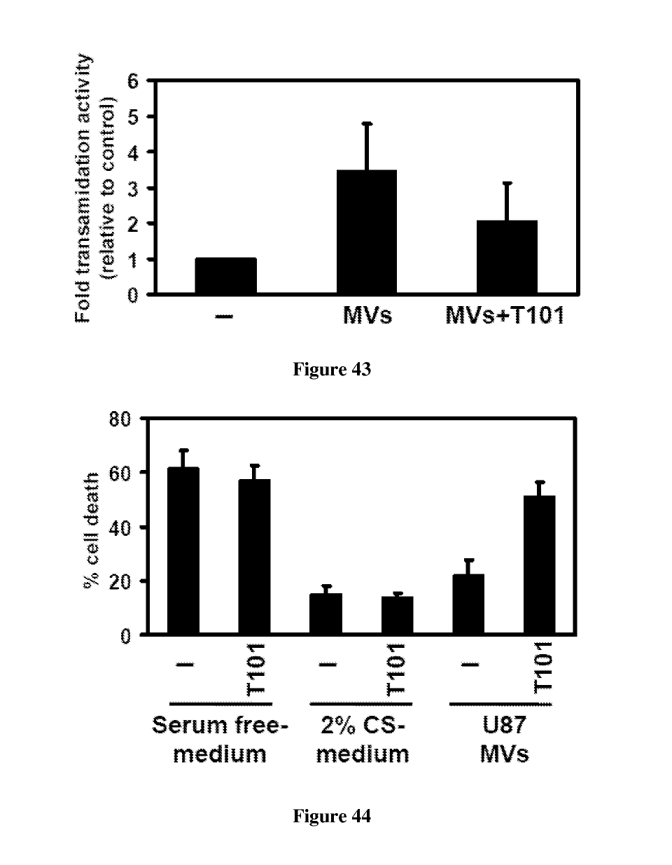

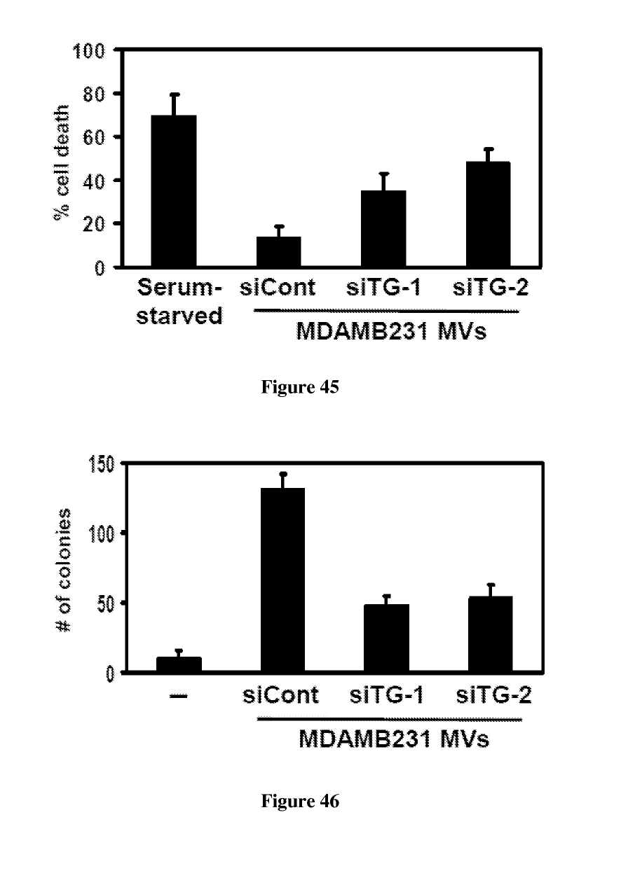

These findings then raised the question of whether the MV-mediated transfer of activated tTG into recipient fibroblasts might be important for conferring these cells with enhanced survival capability and the characteristics of transformation. Taking advantage of our findings that tTG is localized on the surfaces of MVs such that its crosslinking activity is susceptible to inhibition by the cell impermeable, irreversible inhibitor T101 (see FIGS. 13, 16 and 17). By pre-treating cancer cell-derived MVs with T101 before adding them to fibroblast cultures, selectively and irreversibly inhibition of the crosslinking activity of the MV-associated tTG was determined (FIGS. 19 and 43). Using this approach, compared was how the survival advantage afforded to NIH3T3 fibroblasts by MVs collected from cancer cells would be affected under conditions where tTG activity was inhibited. FIGS. 20 and 44 show that pre-treatment of the MVs derived from either MDAMB231 or U87 cells with T101 severely compromised their ability to protect the recipient fibroblasts from serum-deprivation-induced cell death. Importantly, the extent of cell survival achieved by culturing NIH3T3 cells in medium supplemented with a nominal amount of calf serum (2% CS) was unchanged by the addition of T101, indicating that the ability of this small molecule inhibitor to abolish the protection afforded by the cancer cell-derived MVs was not due to off-target effects that sensitized the fibroblasts to apoptosis. Analogous experiments were then performed where either MDAMB231 cell-derived MVs were incubated with serum-starved NIH3T3 cells in the presence of the cell permeable tTG inhibitor MDC (FIG. 20), or MVs collected from MDAMB231 cells in which tTG had been knocked-down (see FIG. 39) were added to serum-starved NIH3T3 cells (FIG. 45). Collectively, the results from these experiments point to a critical role for tTG in mediating the survival advantage imparted to fibroblasts by cancer cell-derived MVs.

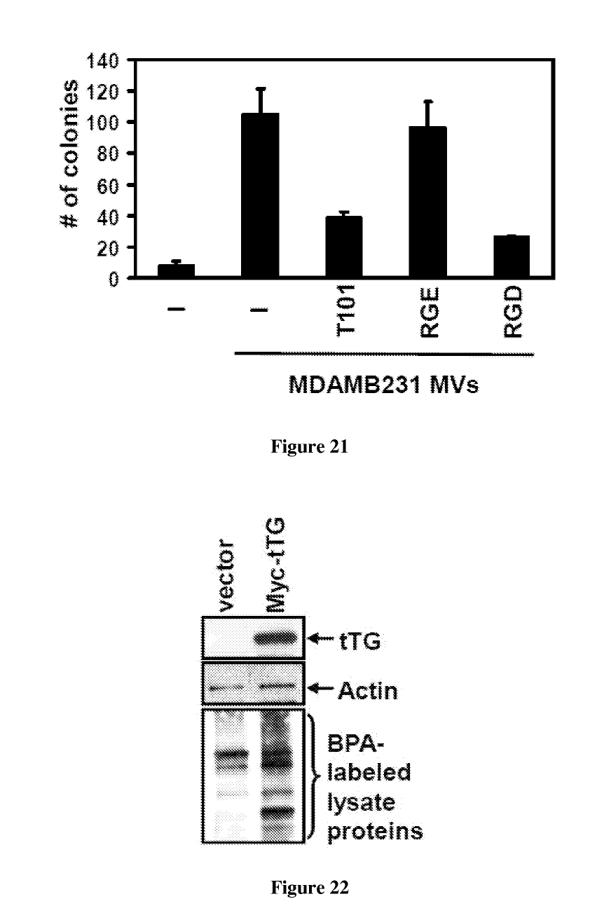

We then tested whether the transforming abilities of the cancer cell-derived MVs were dependent upon tTG. As shown in FIGS. 10 and 33, and again in FIGS. 21, 46 and 47, incubating normal NIH3T3 fibroblasts and MCF10A epithelial cells with MVs derived from MDAMB231 cells or U87 cells induced their ability to grow (i.e. to form colonies) under anchorage-independent conditions. However, when recipient fibroblasts or epithelial cells were incubated with MV preparations that had been pre-treated with T101 (FIGS. 21, 33, and 47), or in which tTG had been knocked-down (FIG. 46), the number of colonies that formed in each case was reduced. It was then verified that T101 did not generally inhibit cellular transformation by showing that this inhibitor had no influence on the ability of NIH3T3 cells expressing an activated form of the small GTPase Cdc42 (Cdc42 F28L) to grow under anchorage-independent conditions, even when a 5-fold excess of T101 was used (FIG. 48).

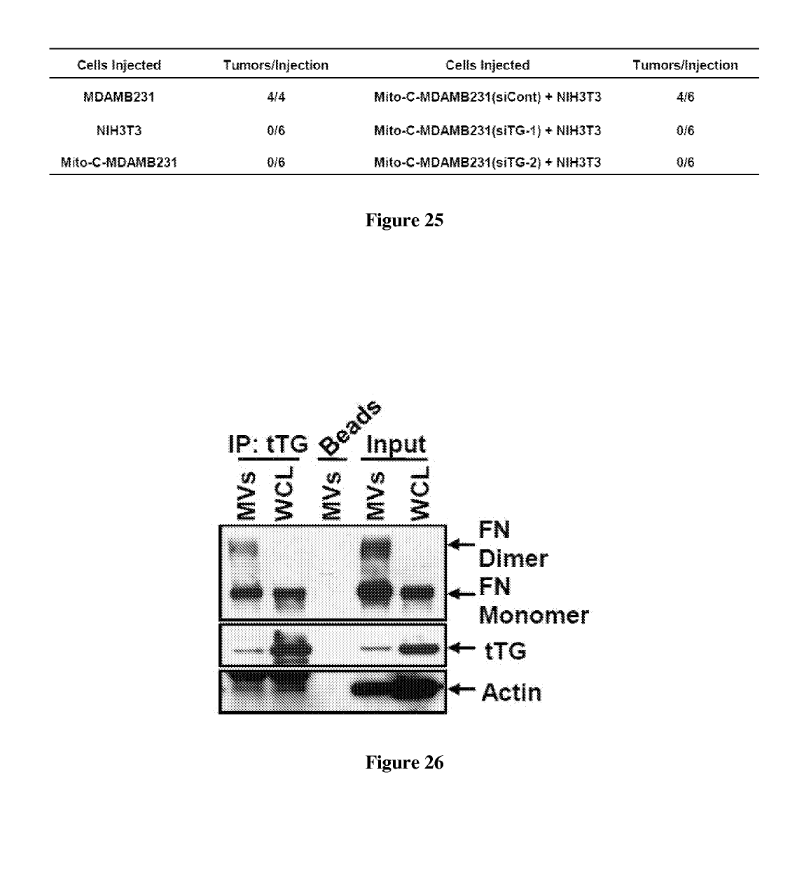

These findings prompted us to then consider whether cancer cell-derived MVs might function similarly in vivo and promote tumor growth by causing normal cells in the tumor microenvironment to acquire the ability to form a tumor. To investigate this, one can take advantage of the fact that exposing MDAMB231 cells to the mitotic arresting agent mitomycin-C, before injecting them into nude mice, inhibited their ability to form tumors under conditions where their control counterparts (untreated MDAMB231 cells) were quite effective at inducing tumor formation (FIG. 25). However, when the mitomycin-C-treated MDAMB231 cells were co-injected with an equal number of normal (non-transformed) NIH3T3 fibroblasts into mice, 4 of 6 mice formed tumors, suggesting that the MVs shed by the mitotically arrested cancer cells were capable of causing the neighboring NIH3T3 fibroblasts to become transformed, inducing tumor growth. Interestingly, it was then shown that knocking-down tTG expression in the mitotically arrested MDAMB231 cells blocked the ability of the co-injected NIH3T3 fibroblasts to form tumors in mice. Thus, these results are consistent with the idea that cancer cells can generate MVs in vivo, and that their ability to cause normal cells in the tumor microenvironment to promote tumor formation is dependent on tTG.

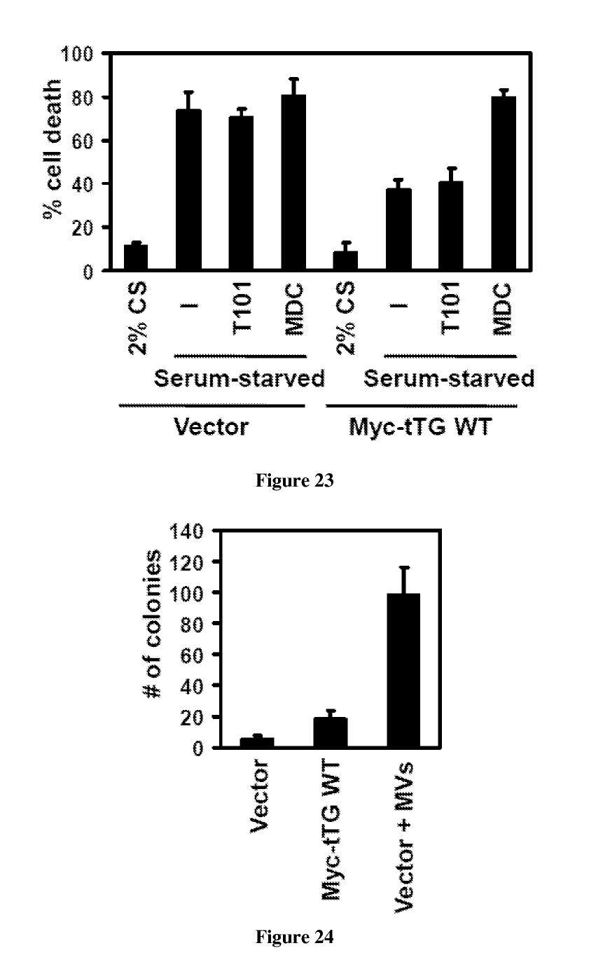

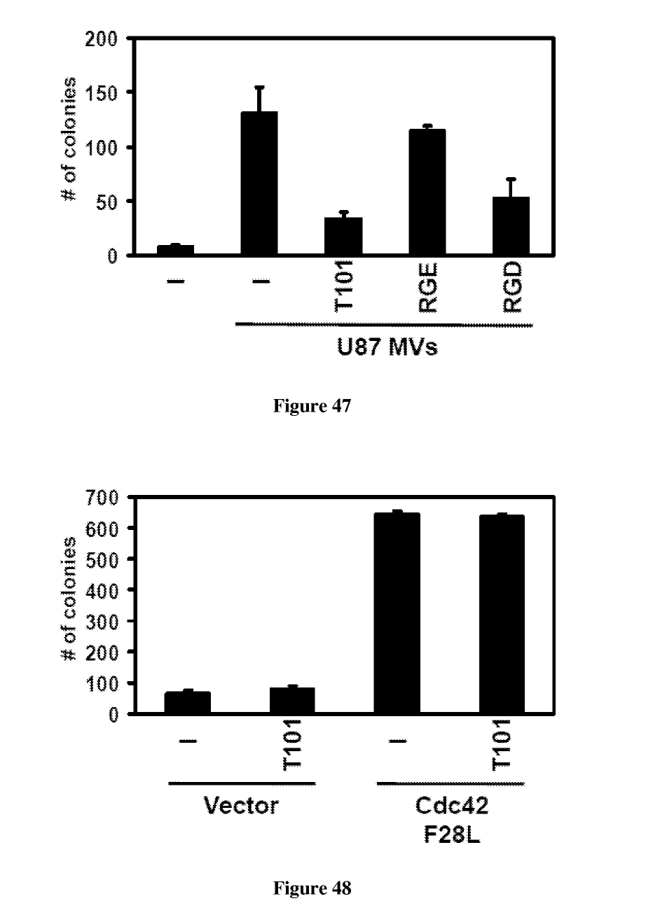

These findings demonstrate that the MV-mediated transfer of tTG into recipient cells is necessary for the ability of MDAMB231 cell- and U87 cell-derived MVs to transform fibroblasts. We then tested whether tTG alone is sufficient to confer survival and transforming capabilities to the recipient cells. We determined that while NIH3T3 fibroblasts stably overexpressing Myc-tagged tTG (FIG. 22) were indeed resistant to serum-deprivation-induced apoptosis, an effect that was ablated by treating the cells with MDC (FIG. 23), they were unable to form colonies in soft agar unlike the case when the vector-control expressing fibroblasts were incubated with MVs derived from MDAMB231 cells (FIG. 24). This means that while over-expression/over-activation of tTG in normal cells is not sufficient to fully induce their transformation (i.e. NIH3T3 cells ectopically expressing tTG do not acquire the ability to form colonies when grown under anchorage-independent conditions), it does confer upon normal cells some characteristics of the transformed state, allowing them to grow in monolayer under low serum conditions, as well as to become less sensitive to serum-deprivation-induced cell death. However, these findings also indicate that in order for cancer cell-derived MVs to enable recipient cells to exhibit one of the major hallmarks of cellular transformation, namely anchorage-independent growth, another protein is likely transferred along with tTG. The cytoskeletal component FN was a particularly attractive candidate, as it is a known binding partner of tTG and was identified in the proteomics screen of MDAMB231 cell- and U87 cell-derived MVs (see Example 3). It was confirmed that FN was expressed in the MVs collected from each of the cancer cell lines by immunoblot analysis (FIG. 12). The potential role of the MV-associated FN in conferring upon fibroblasts the ability to exhibit anchorage-independent growth, by using the RGD-peptide as a means to interfere with the ability of FN to bind to and activate integrins on the surface of the recipient fibroblasts was then assessed. Anchorage-independent growth assays performed on fibroblasts co-treated with MVs derived from MDAMB231 or U87 cells, and either the RGD-peptide or the control RGE-peptide, showed that the RGD-peptide, like T101, blocked the MV-triggered induction of cellular transformation, whereas the control peptide did not (FIGS. 21 and 47).

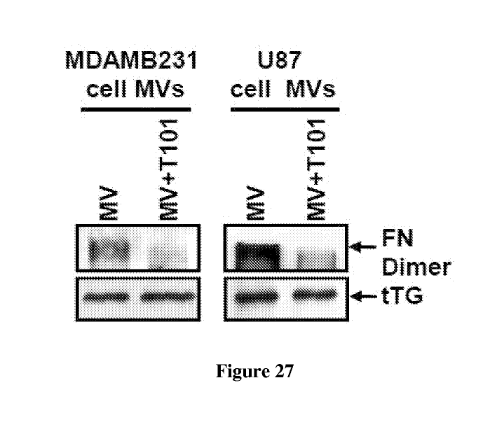

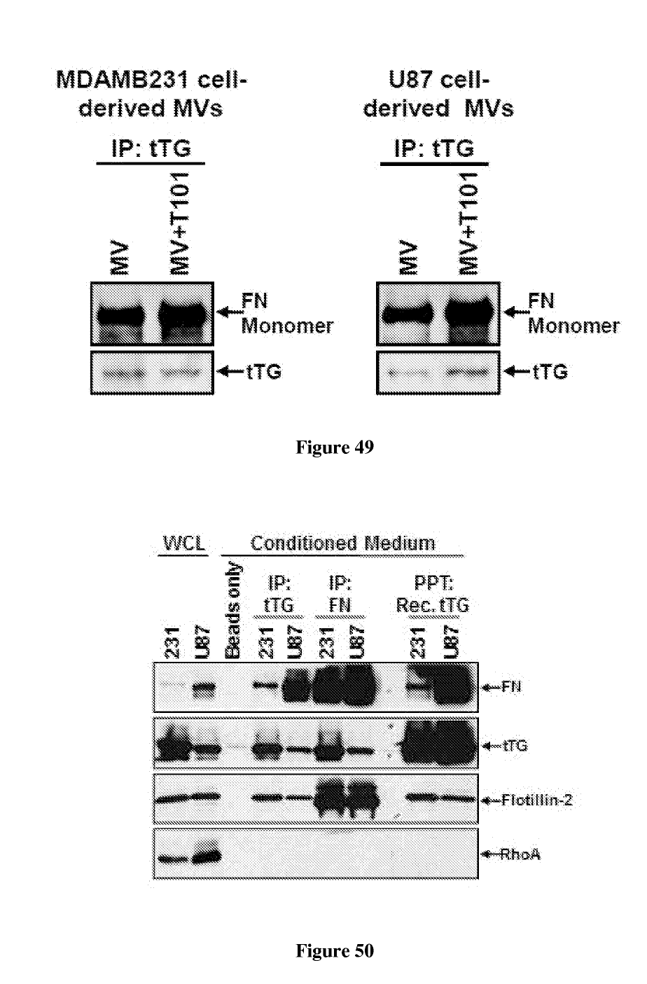

Since both tTG and FN are important for the ability of cancer cell-derived MVs to transform recipient cells, we tested whether they might work together to elicit this cellular outcome. To address this it was first examined whether tTG interacted with FN in MVs. FIG. 26 shows that FN co-immunoprecipitates with tTG from MDAMB231 whole cell lysates as previously reported, as well as from lysates of MVs shed by these cells. In addition to binding the monomeric form of FN, tTG also associated with a larger form of FN with an apparent Mᵣ of ˜440 kDa that likely represented crosslinked FN dimers and was detectable only in the MV lysate. Pre-treating intact MVs collected from MDAMB231 cells or U87 cells with the tTG inhibitor T101, prior to lysing the MVs and subjecting the extracts to immunoblot analysis, did not affect the ability of tTG to be co-immunoprecipitated with monomeric FN from the MV lysates (FIG. 49). However, pre-treating the MVs with the tTG inhibitor resulted in a marked reduction in the amount of the ˜440 kDa FN species detected in the MV lysate samples (FIG. 27), suggesting that the higher molecular mass form of FN in the cancer cell-derived MVs is generated through the ability of tTG to interact with and crosslink FN.

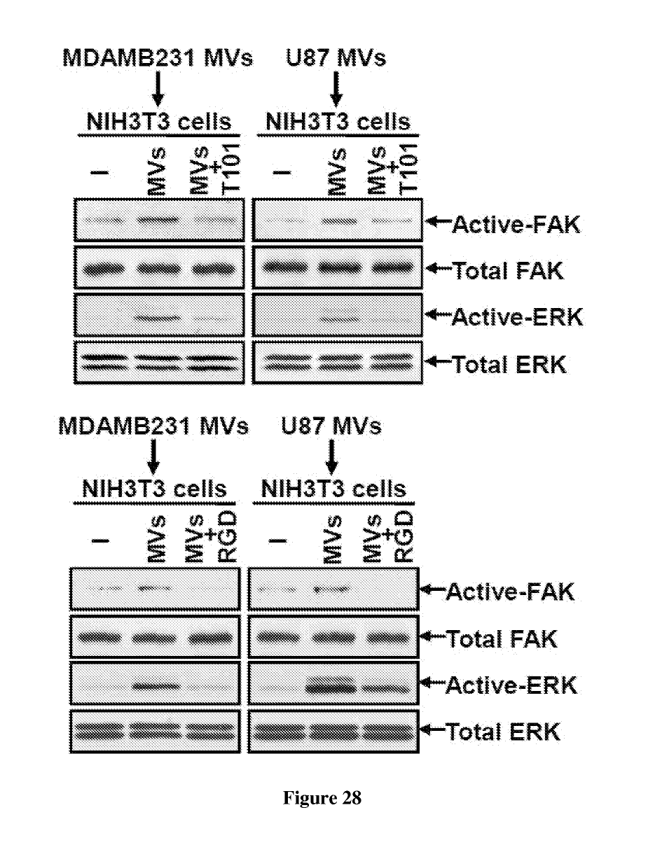

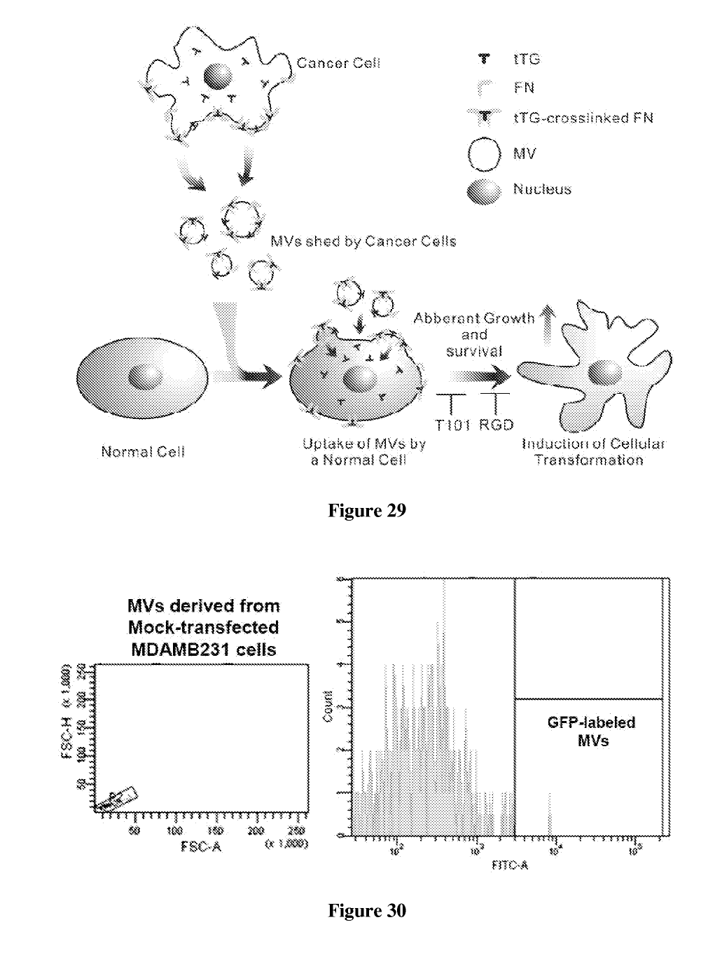

The dimerization of FN strongly enhances its ability to bind and activate integrins on the surfaces of cells. The ability of tTG to crosslink FN in MVs shed by cancer cells suggested that this covalently modified form of FN is capable of potentiating integrin activation. Indeed, it was found that preparations of intact MVs isolated from the medium of serum-deprived MDAMB231 cells or U87 cells were capable of stimulating signaling activities that are well-known to be downstream from activated integrins including FAK and ERK (FIG. 28). Moreover, the activation of these kinases by MVs was blocked either by using the tTG inhibitor T101 or the RGD peptide that interferes with integrin-signaling, thus further demonstrating the importance of tTG and FN for the signaling functions of MVs.

### Example 2

This Example presented a description of proteins common to both MDAMB231 cell- and U87 cell-derived MVs. Proteomic analysis was performed on MVs shed by either MDAMB231 breast cancer cells or U87 brain tumor cells. The following list was compiled (based on general cellular function) of those proteins that were identified in the MVs from both MDAMB231 and U87 cells: Proteomic analyses of microvesicles shed by MDAMB231 cells and U87 cells: Nucleic Acid-binding Proteins; eukaryotic translation elongation factor 1; eukaryotic translation elongation factor 2; histone cluster 1; histone cluster 2; RuvB-like protein 1; RuvB-like protein 2; Extracellular Matrix and Plasma Membrane-associated Proteins; annexin A2; CD9 antigen; collagen; Ecto-5′-nucleotidase; EGF-like repeats and discoidin I-like domains-containing protein 3; fibronectin; galectin 3 binding protein; integrin beta 1; laminin; lysyl hydroxylase precursor; major histocompatibility complex; Na⁺/K⁺-ATPase; transglutaminase 2 isoform a; Metabolic Proteins; aldolase A; enolase; ferritin; glyceraldehyde-3-phosphate dehydrogenase; L-lactate dehydrogenase A; nicotinamide phosphoribosyltransferase precursor; phosphoglycerate kinase 1; pyruvate kinase; UDP-glucose pyrophosphorylase; Cytoskeletal Proteins; actin; actinin; chaperonin; moesin; T-complex protein 1; tubulin; vimentin;

Signaling, Trafficking, and other functional proteins; adenylyl cyclase-associated protein; alpha-2-macroglobulin precursor; heat shock protein 70 kDa; heat shock protein 90 kDa; HtrA serine peptidase 1 precursor; valosin-containing protein.

### Example 3

This Example provides a description of the materials and methods used to present the results described in Example 1.

Materials.

4,6-diamidino-2-phenylindole (DAPI), brefelden A, mitomycin-C, and Exo1 were obtained from Calbiochem, while T101 was from Zedira. The rhodamine-conjugated phalloidin, EGF, Lipofectamine, Lipofectamine 2000, protein G beads, control and tTG siRNAs, and all cell culture reagents were from Invitrogen. The FN antibody, MDC, and BPA were from Sigma. The tTG and actin antibodies were obtained from Lab Vision/Thermo. Flotillin-2 antibody was obtained from Santa Cruz, and HA and Myc antibodies were from Covance. The Steriflip PVDF-filters (0.45 μm pore size) were from Millipore. The antibodies against IκBα, GFP, as well as antibodies that recognize ERK, AKT, FAK and the EGF receptor were from Cell Signaling.

Cell Culture.

The MDAMB231, U87, MCF10A, and HeLa cell lines were grown in RPMI 1640 medium containing 10% fetal bovine serum, while the NIH3T3 cell line was grown in DMEM medium containing 10% calf serum. Expression constructs were transfected into cells using Lipofectamine, whereas the control and tTG siRNAs were introduced into cells with Lipofectamine 2000. As indicated, cells were incubated with serum-free medium containing combinations of 0.1 μg/ml EGF, 100 μM MDC, 10 μM T101, 10 μM BFA, and 10 μM Exo1. To mitotically arrest MDAMB231 cells, plates of cells were treated with 10 μg/ml mitomycin-C for 2 hours, before rinsing the solution away and allowing the cells to recover in growth medium (RPMI-1640 medium containing 10% FBS) for a day.

Isolation of Microvesicles from Cancer Cells.

For each of the experiments that used MV preparations, the conditioned medium from 5.0×10⁶ serum-starved MDAMB231 cells or U87 cells (which is the equivalent of two nearly confluent 150 _{MM} dishes of either of these cell lines) were collected and the MVs isolated from the medium as previously described. Briefly, the conditioned medium was subjected to two consecutive centrifugations; the first at 300 g for 10 minutes pelleted intact cells, while the second at 12,000 g for 20 minutes pelleted cell debris. To generate MV lysates, the conditioned medium was centrifuged a third time at 100,000 g for 2 hours and the resulting pellet was washed with PBS and then lysed in 250 μl cell lysis buffer (25 mM Tris, 100 mM NaCl, 1% Triton X-100, 1 mM EDTA, 1 mM DTT, 1 mM NaVO₄, 1 mM β-glycerol phosphate, and 1 μg/mL aprotinin). To generate intact MVs for the cell-based assays and other experiments as indicated, the partially purified conditioned medium (medium cleared of cells and cell debris) was then filtered using a Millipore Steriflip PVDF-filter with a 0.45 μm pore size. The microvesicles retained by the PVDF membrane were then resuspended in serum-free medium, with each preparation yielding enough MVs to treat 2-3 wells of a 6-well dish of recipient cells for a given experiment.

Immunoblot Analysis and Immunoprecipitation.

The protein concentrations of the whole cell lysates (WCLs) were determined using the Bio-Rad DC protein assay, while the MV lysates were normalized for comparison by isolating them from the conditioned medium of 5.0×10⁶ serum-starved MDAMB231 cells or U87 cells for each of the experimental conditions assayed, and then lysing in 250 μl of cell lysis buffer. For immunoprecipitations, equal volumes of MV lysates, or 300 μg of WCLs, were incubated with a tTG antibody and protein G beads. In certain instances the medium from cells was incubated with tTG or fibronectin antibodies and protein G beads. The bead-antibody-protein complexes collected by centrifugation, as well as the WCLs (40 μg) and MV extracts (75 μl), were resolved by SDS-PAGE and the proteins transferred to polyvinylidene difluoride membranes. The filters were incubated with the indicated primary antibodies diluted in TBST (20 mM Tris, 135 mM NaCl, and 0.02% Tween 20). The primary antibodies were detected with horseradish peroxidase-conjugated secondary antibodies (Amersham Biosciences) followed by exposure to ECL reagent.

Immunofluorescence.

Cells were fixed with 3.7% paraformaldehyde and then some samples were permeabilized with phosphate-buffered saline (PBS) containing 0.1% Triton X-100. Permeabilized and non-permeabilized samples were incubated with a tTG antibody, and then incubated with Oregon green 488-conjugated secondary antibody. Rhodamine-conjugated phalloidin was used to stain actin and DAPI was used to stain nuclei. The cells were visualized by fluorescent microscopy and the images were captured and processed using IPLABS.

Live Image Fluorescence Microscopy.

MDAMB231 cells transiently expressing GFP-PM, a GFP-tagged form of the plasma membrane targeting sequence in Lyn, were visualized by fluorescent microscopy. Images of the transfectants were captured in 30 second intervals over a span of 15 minutes.

Transamidation Assay.

The transamidation activity in whole cell extracts was readout by the incorporation of BPA into lysate proteins as previously described, whereas the transamidation activity of recombinant tTG (0.1 μM) exposed to increasing concentrations of T101 was determined using a spectrophotometric assay. The transamidation activity associated with MVs was readout by incubating equal amounts (75 μl) of each MV sample in a buffer containing 40 mM N′N-dimethyl casein, 2 mM BPA, 40 mM CaCl₂, and 40 mM dithiothreitol for 15 minutes. The reaction was stopped by the addition of Laemmli sample buffer followed by boiling. The reactions were then resolved by SDS-PAGE and the proteins transferred to polyvinylidene difluoride membranes. The filters were blocked with BBST (100 mM boric acid, 20 mM sodium borate, 0.01% SDS, 0.01% Tween 20, and 80 mM NaCl) containing 10% bovine serum albumin, and then incubated with horseradish peroxidase-conjugated streptavidin diluted in BBST containing 5% bovine serum albumin for 1 hour at room temperature, followed by extensive washing with BBST. The incorporation of BPA into N′N-dimethyl casein was visualized after exposing the membranes to ECL reagent.

Scanning Electron Microscopy (SEM) and Immuno-SEM.

MDAMB231 cells grown on Lab-Tek chamber slides (Nunc) were fixed for 1 hour with 2% EM-grade glutaraldehyde diluted in a 0.05 M Cacodylic acid buffer solution (PH=7.4). For immuno-SEM, filter-isolated MDAMB231 cell-derived MVs were added to Lab-Tek chamber slides, allowed to attach, and then were fixed for 1 hour with 2% EM-grade glutaraldehyde in PBS. Following blocking for 15 minutes in 0.1M glycine, and for an additional 30 minutes in PBS containing 5% BSA, 0.1% gelatin and 5% goat serum, the MVs were incubated for 1 hour with the tTG antibody diluted in PBS (2 μg/mL). After washing with PBS, the MV samples were incubated for 1 hour with 6 nm gold particle-conjugated Goat-anti-Mouse IgG (Electron Microscopy Sciences) diluted in PBS. Both the cell and the immuno-labeled MV samples were post-fixed for 1 hour with 1% osmium tetroxide in PBS and dehydrated in graded ethanol solutions of 25%, 50%, 70%, 95%, and 100% ethanol, before being placed in a CPD-30 critical point drying machine (BAL-TEC SCD050). The cells were then sputter-coated with platinum, whereas the immuno-labeled MVs were sputter-coated with amorphous carbon, before being observed with a Leo 1550 Field-Emission Scanning Electron Microscope.

Cell Growth Assays.

NIH3T3 cells were plated in each well of a 6-well dish at a density of 10×10⁴ cells/well and were maintained in DMEM medium containing 2% CS supplemented without or with MVs derived from 5.0×10⁶ MDAMB231 cells or U87 cells. Once a day for three days, one set of cultures was collected and counted, while the remaining sets of cells had their culturing medium replenished (including the addition of freshly isolated MVs). The assays were performed three times and the results were averaged together and graphed.

Anchorage-independent Growth Assays.

Parental NIH3T3 cells or MCF10A cells incubated without or with MVs derived from 5.0×10⁶ MDAMB231 cells or U87 cells, or NIH3T3 cells stably overexpressing the vector-control, wild-type tTG, or Cdc42 F28L, were plated at a density of 7×10³ cells/ml in medium containing 0.3% agarose, without or with various inhibitors as indicated, onto underlays composed of growth medium containing 0.6% agarose in six-well dishes. The soft agar cultures were re-fed (including the addition of freshly prepared MVs and treatments with various inhibitors as indicated) every third day for 12 days, at which time the colonies that formed were counted. Each of the assays was performed at least three times and the results were averaged together and graphed.

Cell Death Assays.

NIH3T3 cells or MCF10A cells were plated in each well of a 6-well dish and then cultured in medium containing 2% CS or serum-free medium supplemented without or with MVs derived from 5.0×10⁶ MDAMB231 cells or U87 cells, and without or with MDC or T101, as indicated. Two days later the cultures were fixed and stained with DAPI for viewing by fluorescence microscopy. Cells undergoing apoptosis were identified by nuclear condensation or blebbing and the percentage of cell death was determined by calculating the ratio of apoptotic to total cells for each condition. These experiments were conducted at least three times and the results from each experiment were averaged together and graphed.

Flow Cytometry.

Intact MVs were isolated from the conditioned medium of 5.0×10⁶ mock transfected MDAMB231 cells or MDAMB231 cells transiently expressing GFP using the filter method (described above) and re-suspended in PBS containing 0.1% BSA. The MV samples were evaluated using a BD LSR II flow cytometer by gating events that were between ˜1-3 μm in size and determining whether they expressed GFP. At least 500 events were collected for each sample and then the data was analyzed using BD FACSDiva software. The experiments were performed at least three times, with similar results being obtained from each experiment.

Proteomic Analyses.

Lysates of MDAMB231 cell- and U87 cell-derived MVs (˜30 μg of each sample) were resolved by SDS-PAGE and then stained using the Colloidal Blue Staining Kit (Invitrogen) according to the manufacturer's protocol. The proteins were excised from the gel and then digested with trypsin. The resulting protein samples were analyzed at Cornell Proteomic Facility using a 4000 Q Trap (Triple quadrupole linear ion trap) On-line LC/MS/MS system (Applied Biosystems/MDS Sciex) or Synapt HDMS system (Waters). Protein identification was achieved by performing peptide alignment searches against the NCBI refseq protein database.

Mouse Studies.

5×10⁵ mitotically arrested (using mitomycin-C) MDAMB231 cells stably expressing control or tTG siRNAs were combined with 5×10⁵ NIH3T3 fibroblasts and growth factor-reduced Matrigel (BD Biosciences) to achieve 30% Matrigel in the final solution. The cell preparations were subcutaneously injected into the flanks of 6-8 weeks-old female NIH-III nude mice. As controls, parental MDAMB231 cells and NIH3T3 cells (5×10⁵ cells of each cell line) were singly combined with growth factor-reduced Matrigel (to a final concentration of 30% Matrigel) and then were injected into mice as well. After a month, the animals were sacrificed and the resulting tumors that formed for each experimental condition were excised and counted. The experiments involving mice were performed in accordance with the protocols approved by The Cornell Center for Animal Resources and Education (CARE).

To obtain the results depicted in FIG. 51, the following procedures were used. In vitro liposome fractionation assays—Synthetic liposomes were prepared from a lipid mixture containing 35% phosphatidylethanolamine, 25% phosphatidylserine, 5% phosphatidylinositol, and 35% cholesterol re-suspended in TBSM buffer (20 mM Tris, pH 7.5, 150 mM NaCl, and 2 mM MgCl₂). The lipids were extruded through an 8 micron filter, pelleted by centrifugation at 13,000 rpm for 15 minutes, and re-suspended in TBSM buffer. Equal amounts of the lipid preparation were then incubated with either recombinant wild-type tTG or BSA for 15 minutes, followed by centrifugation at 13,000 rpm for 10 minutes at room temperature. The supernatant was concentrated to ˜30 μL using a microfuge concentrator with a 10K molecular weight cut-off, while the pelleted liposomes were re-suspended in 30 μL of TBSM buffer. Each of the samples was resolved on a gel and then stained with Quick Blue to detect proteins.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1. A method for characterizing microvesicles, the method comprising:
i) obtaining a sample comprising microvesicles;
ii) assaying the microvesicles for tissue transglutaminase (tTG) and/or cross-linked fibronectin (FN); and
identifying the microvesicles as tTG positive if tTG associated with the microvesicles is present in the sample, and identifying the microvesicles as tTG negative if tTG associated with the microvesicles is absent from the sample, and/or identifying the microvesicles as cross-linked FN positive if cross-linked FN associated with the microvesicles is present in the sample, and identifying the microvesicles as cross-linked FN negative if cross-linked FN associated with the microvesicles is absent from the sample.

2. The method of claim 1, wherein the sample comprises a liquid biological sample from an individual diagnosed with, suspected of having, or at risk for cancer.

3. The method of claim 2, wherein the assaying the microvesicles includes separating the microvesicles from the liquid biological sample by capturing the microvesicles on a binding partner.

4. The method of claim 3, wherein the binding partner is attached to a solid substrate.

5. The method of claim 4, wherein the binding partner is selected from fibronectin, an anti-fibronectin antibody or fibronectin binding fragment thereof, recombinant tTG or a derivative thereof, an anti-tTG antibody, and a tTG binding fragment thereof.

6. The method of claim 2, wherein the individual has been diagnosed with cancer and is undergoing cancer therapy.

7. A method of diagnosing an individual as having circulating microvesicles that are
a) tissue transglutaminase (tTG) positive or tTG negative, and/or
b) cross-linked fibronectin (FN) positive or cross-linked FN negative;
the method comprising:
i) assaying a sample obtained from the individual for tTG associated with microvesicles, and identifying the individual has having circulating tTG associated microvesicles if tTG is present, and identifying the individual as not having circulating tTG associated microvesicles if tTG associated with microvesicles is absent; and/or
ii) assaying the sample for cross-linked FN associated with microvesicles, and identifying the individual has having circulating cross-linked FN associated microvesicles if cross-linked FN is present, and identifying the individual as not having circulating cross-linked FN associated microvesicles if cross-linked FN associated with microvesicles is absent.

8. The method of claim 7, wherein the sample comprises a liquid biological sample from an individual diagnosed with, suspected of having, or at risk for cancer.

9. The method of claim 7, wherein the assaying the sample includes separating the microvesicles from the liquid biological sample by capturing the microvesicles on a binding partner.

10. The method of claim 9, wherein the binding partner is attached to a solid substrate.

11. The method of claim 10, wherein the binding partner is selected from fibronectin, an anti-fibronectin antibody or fibronectin binding fragment thereof, recombinant tTG or a derivative thereof, an anti-tTG antibody, and tTG binding fragments thereof.

12. The method of claim 7, wherein the individual has been diagnosed with cancer and is undergoing cancer therapy.

13. A method for inhibiting in an individual transfer of cargo from microvesicles which comprises tissue transglutaminase (tTG) to one or more cells in the individual comprising administering to the individual a cell-impermeable tTG inhibitor.

14. The method of claim 13, wherein the individual has been diagnosed with, is suspected of having, or is at risk for cancer.

15. A composition comprising an isolated population of microvesicles, wherein the microvesicles comprise tissue transglutaminase (tTG), wherein the isolated population of microvesicles is attached to a tTG binding partner, or to a recombinant tTG or derivative thereof, or to a cross-linked FN binding partner.

16. The composition of claim 15, wherein the binding partner is attached to a solid substrate.

17. The composition of claim 16, wherein the binding partner is selected from fibronectin, recombinant tTG or a derivative thereof, an anti-tTG antibody, and combinations thereof.

18. A method for isolating membranous structures cells comprising providing a sample which may comprise the membranous structures, mixing the sample with tissue transglutaminase (tTG) or a derivative thereof, and if the membranous structures are present in the sample, allowing formation of a complex of the membranous structures and the tTG or the derivative thereof, and separating the complex of the tTG and the membranous structures from the sample.

19. The method of claim 18, wherein the sample comprises a liquid biological sample from an individual diagnosed with, suspected of having, or at risk for cancer.

20. The method of claim 18, wherein the membranous structures are shed from cells.
